(19) United States
(12) Patent Application Publication
(10) Pub. No.: US 2020/0171141 A1
(43) Pub. Date: Jun. 4, 2020

(54) Compositions and Methods for Generating an Immune Response to LASV

(71) Applicant: GEOVAX, INC., Smyma, GA (US)

(72) Inventors: Farshad Guirakhoo, Atlanta, GA (US); Arban Domi, Sandy Springs, GA (US)

(21) Appl. No.: 16/631,489

(22) PCT Filed: Jul. 18, 2018
(86) PCT No.: PCT/US2018/042645

§ 371 Date: Jan. 16, 2020

Related U.S. Application Data
(60) Provisional application No. 62/533,998, filed on Jul. 18, 2017.

(51) Int. Cl. A61K 39/12; C12N 15/86

(52) U.S. Cl. CPC A61K 39/12; C12N 15/86; C12N 2710/24143; A61K 2039/70; C12N 2760/00034; C12N 2760/00071; C12N 2760/00022

(57) ABSTRACT

Compositions and methods are described for generating an immune response to an arenavirus. The compositions and methods described herein relate to a modified vaccinia Ankara (MVA) vector encoding one or more viral antigens for generating a protective immune response to a member of genus Arenavirus (such as a member of species Lassa virus) in the subject to which the vector is administered. The compositions and methods of the present invention may be used to prevent and/or treat an infection caused by arenavirus.

### CROSS REFERENCE TO RELATED APPLICATIONS

This application claims the benefit of U.S. provisional patent application U.S. 62/533,998 filed Jul. 18, 2017, the disclosures of which are hereby incorporated by reference in their entirety.

### FIELD OF THE INVENTION

The present invention is directed to compositions, including vaccine compositions, for generating an immune response to arenaviruses (members of family Arenaviridae) such as members of genus Arenavirus. More specifically, the compositions and methods described herein relate to a modified vaccinia Ankara (MVA) vector encoding one or more viral antigens for generating a protective immune response in the subject to which the vector is inhibited to a member of genus Arenavirus (such as a member of species Lassa virus). The compositions and methods of the present invention are useful both prophylactically and therapeutically.

### BACKGROUND OF THE INVENTION

Arenaviridae comprises a family of viruses whose members are generally associated with rodent-transmitted diseases in humans. Arenaviruses are divided into two groups: the New World or Tacaribe complex and the Old World or LCM/Lassa complex. Arenavirus infections are relatively common in humans in some areas of the world and can cause severe illnesses.

Lassa virus (LASV) is an arenavirus that causes Lassa hemorrhagic fever, a type of viral hemorrhagic fever (VHF), in human and non-human primates. Lassa hemorrhagic fever surpasses Ebola, Marburg, and all other hemorrhagic fevers except Dengue in its negative impact on public health. Caused by Lassa virus (LASV; family Arenaviridae, species Lassa mammarenavirus), the disease has a grievous impact, with 100,000-300,000 persons infected each year resulting in 5,000-10,000 deaths annually in West Africa (McCormick, J. B., et al., J Infect Dis 155, 8 (1987); McCormick, J. B. et al., in Emergence and control of rodent-borne viral diseases (eds. Saluzzo, J. F. & Dodet, B.) 177-195 (Elsevier, Amsterdam, 1999)). Analysis of the seroepidemiologic data suggests that the number of cases might be much higher, reaching 3 million cases and 67,000 fatalities per year, putting as many as 200 million persons at risk of infection (Richmond, J. K., BMJ 327, 5 (2003). LASV is zoonotic and is shed in urine and droppings from its reservoir host, predominantly the multimammate rat (Mastomys natalensis), which is found throughout sub-Saharan Africa. Discovery of new species of rodent hosts for LASV might have implications in wider distribution of the virus throughout new areas in West Africa (Olayemi, A., et al., Scientific reports 6 (2016)). Virus is mainly transmitted to humans by direct consumption in food, by contact with rodent urine and droppings, or by inhalation of virus particles from the excreta of infected animals, but can also be transmitted from human to human through nosocomial infections (Fisher-Hoch, S. P. et al., BMJ 311, 3 (1995)). The virus is co-endemic in areas of high HIV prevalence and consequently any medical countermeasures (MCM) against LASV will need to be applicable and safe for immunocompromised individuals. There are four major (I-IV) lineages and one minor emerging lineage (V) (Manning, J. T., et al. Frontiers in microbiology 6 (2015)) that are genetically distinct from one another. Like smallpox and anthrax, LASV is considered a “category A” biological weapon agent because it has the potential to cause widespread illness and death and no effective and practical MCM are available to protect individuals living in or traveling to endemic areas (Shaffer, J. G., PLoS Negl Trop Dis 80 (2014); Asogun, D. A., et al., PLoS Negl Trop Dis 6 (2012)). Ribavirin has been used to treat patients, but it is only effective if given early in the course of illness.

Currently there is no US licensed vaccine for humans against the LASV. Lassa fever is one of the most prevalent viral hemorrhagic fevers in West Africa responsible for thousands of deaths annually.

What is therefore needed are vaccine compositions and methods of use to prevent and treat disease caused by LASV infection.

### SUMMARY OF THE INVENTION

The compositions and methods of the invention described herein are useful for generating an immune response to at least one hemorrhagic fever virus in a subject in need thereof. Advantageously, the compositions and methods may be used prophylactically to immunize a subject against Lassa virus infection, or used therapeutically to prevent, treat or ameliorate the onset and severity of disease.

In a first aspect, the present invention is a recombinant modified vaccinia Ankara (MVA) vector comprising a) a Lassa virus glycoprotein sequence selected from either a stabilized prefusion glycoprotein or deglycosylation mutant glycoprotein sequence and b) a matrix protein sequence, wherein both the glycoprotein sequence and matrix protein sequence are inserted into the MVA vector under the control of promoters compatible with poxvirus expression systems.

In one embodiment, the deglycosylation mutant glycoprotein sequence comprises mutation N99D.

In one embodiment, the deglycosylation mutant glycoprotein sequence comprises mutation N119D.

In one embodiment, the deglycosylation mutant glycoprotein sequence comprises mutation N99D and N119D.

In one embodiment, the deglycosylation mutant glycoprotein sequence comprises mutation N390D.

In one embodiment, the deglycosylation mutant glycoprotein sequence comprises mutation N395D.

In one embodiment, the deglycosylation mutant glycoprotein sequence comprises mutations N390D and N395D

In one embodiment, the prefusion glycoprotein is stabilized by introducing a disulphide bridge and changing RRRLL to RRRR.

In one embodiment, the prefusion glycoprotein sequence comprises mutations R2070, G3060 and E329P.

In one embodiment, the prefusion glycoprotein sequence comprises mutations R2070, G3060, E329P and N99D.

In one embodiment, the prefusion glycoprotein sequence comprises mutations R2070, G3060, E329P, and N119D.

In one embodiment, the prefusion glycoprotein sequence comprises mutations R2070, G3060, E329P, N99D and N119D.

In one embodiment, the prefusion glycoprotein sequence comprises mutations R2070, G3060 and E329P, N390D.

In one embodiment, the prefusion glycoprotein sequence comprises mutations R2070, G3060 and E329P, N395D

In one embodiment, the prefusion glycoprotein sequence comprises mutations R2070, G3060 and E329P, N390D and N395D.

In one embodiment, the glycoprotein sequence and the matrix protein sequence are inserted into one or more deletion sites of the MVA vector.

In one embodiment, the glycoprotein sequence and the matrix protein sequence are inserted into the MVA vector in a natural deletion site, a modified natural deletion site, or between essential or non-essential MVA genes.

In another embodiment, the glycoprotein sequence and the matrix protein sequence are inserted into the same natural deletion site, a modified natural deletion site, or between the same essential or non-essential MVA genes

In another embodiment, the glycoprotein sequence and the matrix protein sequence are inserted into a deletion site selected from I, II, III, IV, V or VI and the matrix protein sequence is inserted into a deletion site selected from I, II, III, IV, V or VI.

In another embodiment, the glycoprotein sequence and the matrix protein sequence are inserted into different natural deletion sites, modified deletion sites, or between different essential or non-essential MVA genes.

In a particular embodiment, the matrix protein sequence is a Z sequence from a Lassa virus.

In one embodiment, the matrix sequence is VP40 selected from a filovirus species selected from the group consisting of Zaire ebolavirus, Sudan ebolavirus, Taï forest ebolavirus, Bundibugyo ebolavirus, Reston ebolavirus, and Marburg marburgvirus, or a combination thereof.

In a particular embodiment, the VP40 sequence are from a Zaire ebolavirus.

In a particular embodiment, the VP40 sequence are from a 2014 epidemic strain of Zaire ebolavirus.

In a particular embodiment, the VP40 sequence is from a Sudan ebolavirus.

In a particular embodiment, the VP40 sequence is from Bundibugyo ebolavirus.

In a particular embodiment, the VP40 sequence is from a Zaire ebolavirus.

In a particular embodiment, the VP40 sequence are from a Marburg marburgvirus.

In another embodiment, the glycoprotein sequence is inserted in a first deletion site and matrix protein sequence is inserted into a second deletion site.

In a particular embodiment, the glycoprotein sequence is inserted between two essential and highly conserved MVA genes; and the matrix protein sequence is inserted into a restructured and modified deletion III.

In one embodiment, the deletion III is modified to remove non-essential sequences and insert the matrix protein sequence between essential genes.

In a particular embodiment, the matrix protein sequence is inserted between MVA genes, I8R and G1 L.

In a particular embodiment, the glycoprotein sequence is inserted between two essential and highly conserved MVA genes to limit the formation of viable deletion mutants.

In a particular embodiment, the glycoprotein protein sequence is inserted between MVA genes, I8R and G1 L.

In one embodiment, the promoter is selected from the group consisting of Pm2H5, Psyn II, and mH5 promoters or combinations thereof.

In one embodiment, the recombinant MVA viral vector expresses prefusion glycoprotein and matrix proteins that assemble into VLPs.

In a particular embodiment, the glycoprotein sequence and the matrix protein sequence are from a Lassa virus.

In one embodiment, the recombinant MVA viral vector expresses Lassa virus prefusion glycoprotein and Z proteins that assemble into VLPs.

In one embodiment, the recombinant MVA viral vector expresses Lassa virus glycoprotein, NP and Z proteins that assemble into VLPs.

In a second aspect, the present invention is a pharmaceutical composition comprising the recombinant MVA vector described herein and a pharmaceutically acceptable carrier.

In one embodiment, the recombinant MVA vector is formulated for intraperitoneal, intramuscular, intradermal, epidermal, mucosal or intravenous administration.

In a third aspect, the present invention is a pharmaceutical composition comprising a first recombinant MVA vector and a second recombinant MVA vector, each comprising a glycoprotein sequence selected from either a stabilized prefusion glycoprotein or deglycosylation mutant glycoprotein sequence and a matrix protein sequence, wherein (i) the glycoprotein sequence of the first recombinant MVA vector is different than the glycoprotein sequence of the second recombinant MVA vector and/or (ii) the matrix protein sequence of the first recombinant MVA vector is different than the matrix protein sequence of the second recombinant MVA vector.

In a particular embodiment, the glycoprotein sequence of the first recombinant MVA vector is different than the glycoprotein sequence of the second recombinant MVA vector.

In a particular embodiment, the glycoprotein sequences of the recombinant MVA vectors are wild type Lassa virus glycoprotein and a prefusion Lassa virus glycoprotein comprising mutations for stabilization in a prefusion state.

In another particular embodiment, the matrix protein sequence of the first recombinant MVA vector is from a different species than the matrix protein sequence of the second recombinant MVA vector.

In a particular embodiment, the matrix protein sequences of the recombinant MVA vectors are from a Zaire ebolavirus and a Lassa virus.

In a particular embodiment, the matrix protein sequences of the recombinant MVA vectors are from a Sudan ebolavirus and a Lassa virus.

In a particular embodiment, the matrix protein sequences of the recombinant MVA vectors are from a Bundibugyo ebolavirus and a Lassa virus.

In a particular embodiment, the matrix protein sequences of the recombinant MVA vectors are from a Marburg marburgvirus and a Lassa virus.

In a fifth aspect, the present invention is a method of inducing an immune response in a subject in need thereof, said method comprising administering the composition of the present invention to the subject in an amount sufficient to induce an immune response.

In one embodiment, the immune response is a humoral immune response, a cellular immune response or a combination thereof.

In a particular embodiment, the immune response comprises production of binding antibodies against Lassa virus.

In a particular embodiment, the immune response comprises production of neutralizing antibodies against Lassa virus.

In a particular embodiment, the immune response comprises production of non-neutralizing antibodies against Lassa virus.

In a particular embodiment, the immune response comprises production of a cell-mediated immune response against Lassa virus.

In a particular embodiment, the immune response comprises production of neutralizing and non-neutralizing antibodies against Lassa virus.

In a particular embodiment, the immune response comprises production of neutralizing antibodies and cell-mediated immunity against Lassa virus.

In a particular embodiment, the immune response comprises production of non-neutralizing antibodies and cell-mediated immunity against Lassa virus.

In a particular embodiment, the immune response comprises production of neutralizing antibodies, non-neutralizing antibodies, and cell-mediated immunity against Lassa virus.

In a sixth aspect, the present invention is a method of preventing a Lassa fever virus infection in a subject in need thereof, said method comprising administering the recombinant MVA vector of the present invention to the subject in a prophylactically effective amount.

In a seventh aspect, the present invention is a method of inducing an immune response in a subject in need thereof, said method comprising administering the recombinant MVA vector of the present invention to the subject in a prophylactically effective amount.

In one embodiment, the immune response is considered a surrogate marker for protection.

In another embodiment, the method induces an immune response to a Lassa virus.

In one embodiment, the subject is exposed to Lassa fever virus, but not yet symptomatic of Lassa fever virus infection. In a particular embodiment, treatment results in prevention of a symptomatic infection.

In another embodiment, the subject was recently exposed but exhibits minimal symptoms of infections.

In another embodiment, the method results in amelioration of at least one symptom of infection.

In another embodiment, the method results in reduction or elimination of the subject's ability to transmit the infection to an uninfected subject.

In another embodiment, the method prevents or ameliorates a Lassa virus infection.

In yet another embodiment, the method prevents or ameliorates infections resulting from more than one species of Lassa virus infections.

In a ninth aspect, the present invention is a method manufacturing a recombinant modified vaccinia Ankara (MVA) vector comprising inserting at least one Lassa virus glycoprotein sequence selected from either a stabilized prefusion glycoprotein or deglycosylation mutant glycoprotein sequence and at least one matrix protein sequence into the MVA vector operably linked to promoters compatible with poxvirus expression systems.

In one embodiment, the matrix sequence is VP40 selected from a filovirus species selected from the group consisting of Zaire ebolavirus, Sudan ebolavirus, Taï forest ebolavirus, Bundibugyo ebolavirus, Reston ebolavirus, and Marburg marburgvirus, or a combination thereof.

In a particular embodiment, the VP40 sequence are from a Zaire ebolavirus.

In a particular embodiment, the VP40 sequence are from a 2014 epidemic strain of Zaire ebolavirus.

In a particular embodiment, the VP40 sequence is from a Sudan ebolavirus.

In a particular embodiment, the VP40 sequence is from Bundibugyo ebolavirus.

In a particular embodiment, the VP40 sequence is from a Zaire ebolavirus.

In a particular embodiment, the VP40 sequence are from a Marburg marburgvirus.

In a particular embodiment, the matrix protein sequence is a Z sequence from a Lassa virus.

In a particular embodiment, the glycoprotein sequence is from a Lassa virus, the matrix protein sequence is a Z sequence from a Lassa virus and further comprises a nucleoprotein (NP) sequence from Lassa virus.

In one embodiment, the recombinant MVA viral vector expresses Lassa virus glycoprotein and matrix proteins that assemble into VLPs.

### BRIEF DESCRIPTION OF THE DRAWINGS

FIG. 1 is a simple line drawing illustrating the design of the MVA vectors.

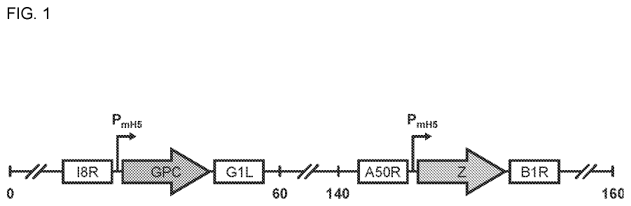

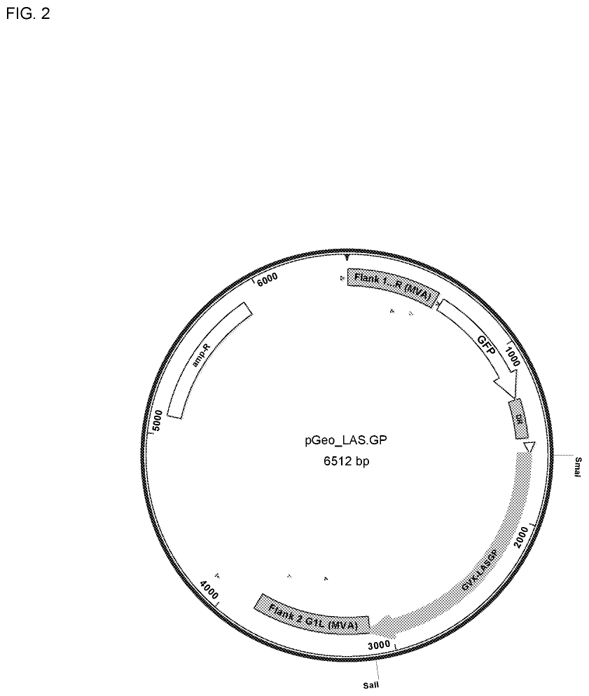

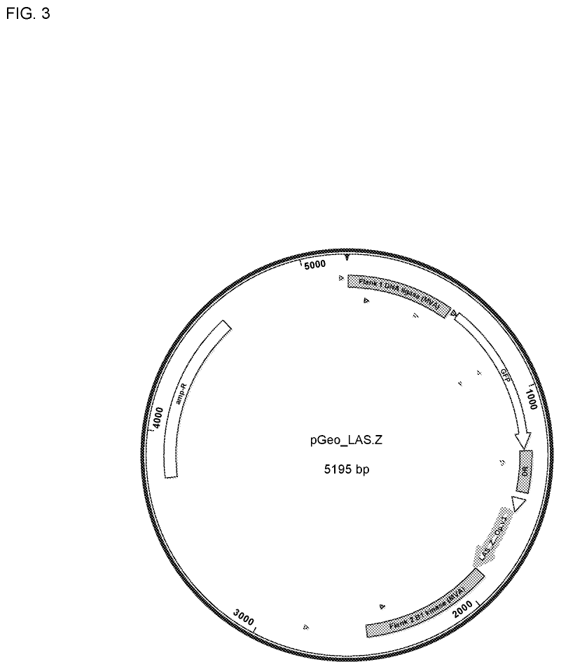

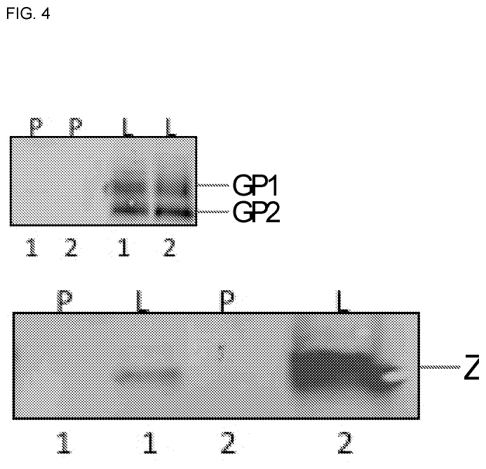

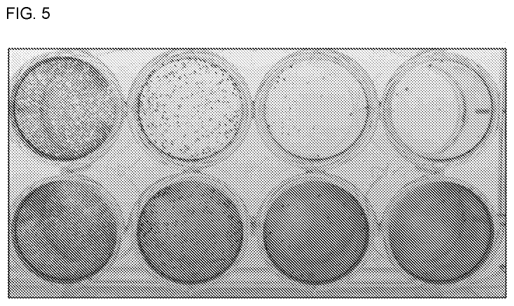

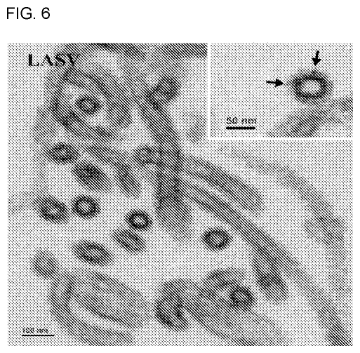

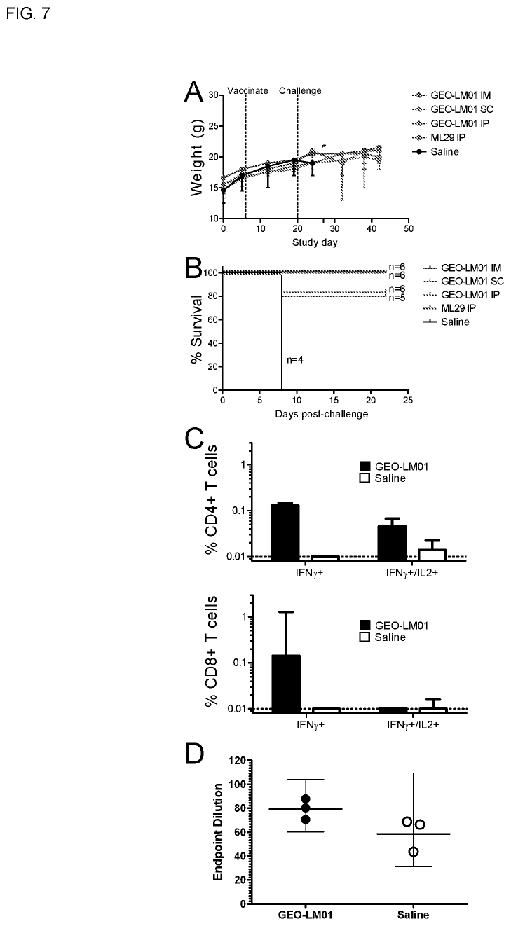

The numbering illustrates the positions (in kilobase pairs) of the various elements in the genome of the MVA vaccine vector. For clarity and brevity, the diagram is not to scale; pairs of diagonal lines indicate a section of the MVA genome that is not illustrated because its contents are not relevant to the invention. Arrows labeled “gpc” and “Z” illustrate the positions of the genes encoding GP and matrix proteins, respectively. In various embodiments, the Z protein may represent another compatible matrix protein described herein. Rectangles labeled “I8R” and “G1L” indicate the positions of the two MVA genetic elements flanking the gene encoding GP. Rectangles labeled “A50R” and “B1R” indicate the positions of the two MVA genetic elements flanking the gene encoding a matrix protein.

LASV Z was inserted into the restructured and modified deletion III and sequences for GPC will be inserted between the I8R and G1L genes. These insertion sites have been identified as supporting high expression and insert stability. P_{mH5}, modified H5 promoter. Numbers are coordinates in the MVA genome.

FIG. 2 is a schematic for the shuttle vector for Lassa virus GP (pGEO_LAS.GP).

The ampicillin resistance marker, allowing the vector to replicate in bacteria, is illustrated with a block labeled “amp-R.” The two flanking sequences, allowing the vector to recombine with the MVA genome, are illustrated with a block and a block labeled “Flank 1” and “Flank 2” respectively. The green fluorescent protein (GFP) selection marker, allowing the selection of recombinant MVAs, is illustrated with an arrow labeled “GFP.” The block labeled “DR” illustrates the location of a sequence homologous to part of Flank 1 of the MVA sequence. DR enables removal of the GFP sequence from the MVA vector after insertion of GP into the MVA genome. The modified H5 (mH5) promoter, which enables transcription of the inserted heterologous gene, is illustrated with a triangle between the DR and GP elements. The Lassa Virus GP gene is illustrated with a grey arrow labeled “GVX-LASGP.”

The shuttle vectors for the various species differ in two principal ways. First, the glycoprotein sequences vary by species. Second, the restriction sites used to insert the glycoprotein sequences into the shuttle vector may vary by species. Neither of these differences affects the orientation of the elements of the shuttle vector.

FIG. 3 provide a schematic for the shuttle vector for Lassa Z genes (pGEO_LAS.Z). The ampicillin resistance marker, allowing the vector to replicate in bacteria, is illustrated with a block labeled “amp-R.” The two flanking sequences, allowing the vector to recombine with the MVA genome, are illustrated with blocks labeled “Flank 1” and “Flank 2.” The green fluorescent protein (GFP) selection marker, allowing the selection of recombinant MVAs, is illustrated with an arrow labeled “GFP.” The block labeled “DR” illustrates the location of a sequence homologous to part of Flank 1 of the MVA sequence. DR enables removal of the GFP sequence from the MVA vector after insertion of Z into the MVA genome. The modified H5 (mH5) promoter enables transcription of the inserted heterologous LASZ gene (LAS_Z . . . Op.v3), is illustrated with a triangle between the DR and LAS_Z elements.

FIG. 4 is a photograph of a Western blot showing expression of LASV GPC and Z proteins. Western blots (WB) for verification of expression of LASV GPC (GP1 and GP2, top) and Z proteins (bottom) in cultured cells. P, Parental (empty) MVA; L, GEO-LM01; 1, cell lysate; 2, supernatant.

FIG. 5 is a photograph showing immunostaining of GEO-LM01 plaques. Immunostaining of GEO-LM01 plaques. Plaques were serially diluted (10-fold) from 1:100 to 1:100,000 and stained with anti-GPC (top row) or anti-Z (bottom row) monoclonal antibodies. Similar plaques numbers at each dilution in the GPC and Z wells show that both inserts are retained.

FIG. 6 is an electron micrograph showing virus-like particle (VLP) production by cells transfected with plasmid DNA vectors encoding Lassa GP and matrix proteins. This experiment demonstrated that antigen sequences of this invention are capable of forming VLPs when introduced into cultured cells.

FIG. 7 provide graphs showing Efficacy and Immunogenicity of GEO-LM01. CBA/J mice vaccinated IM with GEO-LM01 and IP with ML29 showed no appreciable weight loss after IC challenge with ML29 (FIG. 7A) and were 100% protected from death (FIG. 7B). In contrast, sham immunized animals died 8 days after challenge (*) and mice vaccinated SC and IP with GEO-LM01 showed variable survival and weight loss. (FIG. 7C) Antigen-specific CD4 and CD8 T cells were abundant in spleens of immunized animals 11 days after a single IM dose of GEO-LM01, assessed by IFNγ and IL2 expression in response to LASV GP peptides. (FIG. 7D) ML29-specific Ab titers from immunized and mock-immunized mice were not statistically significantly different.

### DETAILED DESCRIPTION OF THE INVENTION

Compositions and methods are provided to produce an immune response to a Lassa fever virus in a subject in need thereof. The compositions and methods of the present invention can be used to prevent infection in an unexposed person or to treat disease in a subject exposed to a Lassa fever virus who is not yet symptomatic or has minimal symptoms. In one embodiment, treatment limits an infection and/or the severity of disease.

Ideal immunogenic compositions or vaccines have the characteristics of safety, efficacy, scope of protection and longevity, however, compositions having fewer than all of these characteristics may still be useful in preventing viral infection or limiting symptoms or disease progression in an exposed subject treated prior to the development of symptoms. In one embodiment the present invention provides a vaccine that permits at least partial, if not complete, protection after a single immunization.

In one embodiment, the composition is a recombinant vaccine that comprises one or more genes from a Lassa fever virus and combinations thereof.

In exemplary embodiments, the immune responses are long-lasting and durable so that repeated boosters are not required, but in one embodiment, one or more administrations of the compositions provided herein are provided to boost the initial primed immune response.

### I. Definitions

Where a term is provided in the singular, the inventors also contemplate aspects of the invention described by the plural of that term. As used in this specification and in the appended claims, the singular forms “a”, “an” and “the” include plural references unless the context clearly dictates otherwise, e.g., “a peptide” includes a plurality of peptides. Thus, for example, a reference to “a method” includes one or more methods, and/or steps of the type described herein and/or which will become apparent to those persons skilled in the art upon reading this disclosure.

The term “antigen” refers to a substance or molecule, such as a protein, or fragment thereof, that is capable of inducing an immune response.

The term “arenavirus” refers to any virus that is a member of the family Arenaviridae.

The term “binding antibody” or “bAb” refers to an antibody which either is purified from, or is present in, a body fluid (e.g., serum or a mucosal secretion) and which recognizes a specific antigen. As used herein, the antibody can be a single antibody or a plurality of antibodies. Binding antibodies comprise neutralizing and non-neutralizing antibodies.

The term ““cell-mediated immune response” refers to the immunological defense provided by lymphocytes, such as the defense provided by sensitized T cell lymphocytes when they directly lyse cells expressing foreign antigens and secrete cytokines (e.g., IFN-gamma), which can modulate macrophage and natural killer (NK) cell effector functions and augment T cell expansion and differentiation. The cellular immune response is the 2^{nd} branch of the adaptive immune response.

The term “conservative amino acid substitution” refers to substitution of a native amino acid residue with a non-native residue such that there is little or no effect on the size, polarity, charge, hydrophobicity, or hydrophilicity of the amino acid residue at that position, and without resulting in substantially altered immunogenicity. For example, these may be substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Conservative amino acid modifications to the sequence of a polypeptide (and the corresponding modifications to the encoding nucleotides) may produce polypeptides having functional and chemical characteristics similar to those of a parental polypeptide.

The term “deglycosylation mutant” refers to a change to the nucleotide sequence that encodes the amino acid Asparagine (Asn=N) to instead encode for another amino acid such as Aspartic acid (Asp=D), thereby preventing glycosylation of the protein in that site. The “glycosylation” term means the attachment of a glycan residue to a protein in the biosynthesis pathway of N-linked glycoproteins. This requires the recognition of a consensus sequence in glycosylation process. N-linked glycans are almost always attached to the nitrogen atom of an Asn (N) side chain that is present as a part of Asn-X-Ser/Thr consensus sequence, where X is any amino acid except proline (Pro) (Dalziel M, Crispin M; Scanlan C N, Zitzmann N, Dwek R A (January 2014). “Emerging principles for the therapeutic exploitation of glycosylation” Science. 343 (6166): 1235681.)

The term “deletion” in the context of a polypeptide or protein refers to removal of codons for one or more amino acid residues from the polypeptide or protein sequence. The term deletion in the context of a nucleic acid refers to removal of one or more bases from a nucleic acid sequence.

The term “fragment” in the context of a proteinaceous agent refers to a peptide or polypeptide comprising an amino acid sequence of at least 2 contiguous amino acid residues, at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a peptide, polypeptide or protein. In one embodiment, a fragment of a full-length protein retains activity of the full-length protein. In another embodiment, the fragment of the full-length protein does not retain the activity of the full-length protein.

The term “fragment” in the context of a nucleic acid refers to a nucleic acid comprising an nucleic acid sequence of at least 2 contiguous nucleotides, at least 5 contiguous nucleotides, at least 10 contiguous nucleotides, at least 15 contiguous nucleotides, at least 20 contiguous nucleotides, at least 25 contiguous nucleotides, at least 30 contiguous nucleotides, at least 35 contiguous nucleotides, at least 40 contiguous nucleotides, at least 50 contiguous nucleotides, at least 60 contiguous nucleotides, at least 70 contiguous nucleotides, at least contiguous 80 nucleotides, at least 90 contiguous nucleotides, at least 100 contiguous nucleotides, at least 125 contiguous nucleotides, at least 150 contiguous nucleotides, at least 175 contiguous nucleotides, at least 200 contiguous nucleotides, at least 250 contiguous nucleotides, at least 300 contiguous nucleotides, at least 350 contiguous nucleotides, or at least 380 contiguous nucleotides of the nucleic acid sequence encoding a peptide, polypeptide or protein. In a preferred embodiment, a fragment of a nucleic acid encodes a peptide or polypeptide that retains activity of the full-length protein. In another embodiment, the fragment encodes a peptide or polypeptide that of the full-length protein does not retain the activity of the full-length protein.

As used herein, the term “GP” refers to the Lassa fever virus surface glycoprotein, or the gene or transcript encoding the Lassa fever virus surface glycoprotein.

As used herein, the phrase “heterologous sequence” refers to any nucleic acid, protein, polypeptide or peptide sequence which is not normally associated in nature with another nucleic acid or protein, polypeptide or peptide sequence of interest.

As used herein, the phrase “heterologous gene insert” refers to any nucleic acid sequence that has been or is to be inserted into the recombinant vectors described herein. The heterologous gene insert may refer to only the gene product encoding sequence or may refer to a sequence comprising a promoter, a gene product encoding sequence (such as GP, VP or Z), and any regulatory sequences associated or operably linked therewith.

The term “homopolymer stretch” refers to a sequence comprising at least four of the same nucleotides uninterrupted by any other nucleotide, e.g., GGGG or TTTTTTT.

The term “humoral immune response” refers to the stimulation of Ab production. Humoral immune response also refers to the accessory proteins and events that accompany antibody production, including T helper cell activation and cytokine production, affinity maturation, and memory cell generation. The humoral immune response is one of two branches of the adaptive immune response.

The term “humoral immunity” refers to the immunological defense provided by antibody, such as neutralizing Ab that can directly block infection; or, binding Ab that identifies a virus or infected cell for killing by such innate immune responses as complement (C′)-mediated lysis, phagocytosis, and natural killer cells.

The term “immune response” refers to any response to an antigen or antigenic determinant by the immune system of a subject (e.g., a human). Exemplary immune responses include humoral immune responses (e.g., production of antigen-specific antibodies) and cell-mediated immune responses (e.g., production of antigen-specific T cells).

The term “improved therapeutic outcome” relative to a subject diagnosed as infected with a particular virus (e.g., an ebolavirus) refers to a slowing or diminution in the growth of virus, or viral load, or detectable symptoms associated with infection by that particular virus; or a reduction in the ability of the infected subject to transmit the infection to another, uninfected subject.

The term “inducing an immune response” means eliciting a humoral response (e.g., the production of antibodies) or a cellular response (e.g., the activation of T cells) directed against a virus (e.g., Lassa fever virus) in a subject to which the composition (e.g., a vaccine) has been administered.

The term “insertion” in the context of a polypeptide or protein refers to the addition of one or more non-native amino acid residues in the polypeptide or protein sequence. Typically, no more than about from 1 to 6 residues (e.g. 1 to 4 residues) are inserted at any one site within the polypeptide or protein molecule.

The term “lassavirus,” “Lassa virus,” or “LASV” refers to an arenavirus that is any member of the species Lassa virus.

The term “modified vaccinia Ankara,” “modified vaccinia ankara,” “Modified Vaccinia Ankara,” or “MVA” refers to a highly attenuated strain of vaccinia virus developed by Dr. Anton Mayr by serial passage on chick embryo fibroblast cells; or variants or derivatives thereof. MVA is reviewed in (Mayr, A. et al. 1975 Infection 3:6-14; Swiss Patent No. 568,392).

The term “neutralizing antibody” or “NAb” is meant an antibody which either is purified from, or is present in, a body fluid (e.g., serum or a mucosal secretion) and which recognizes a specific antigen and inhibits the effect(s) of the antigen in the subject (e.g., a human). As used herein, the antibody can be a single antibody or a plurality of antibodies.

The term “non-neutralizing antibody” or “nnAb” refers to a binding antibody that is not a neutralizing antibody.

The term “prefusion glycoprotein” refers to an expressed Lassa virus glycoprotein monomer where glycoprotein subunits GP1 and GP2 are expressed as a single unit with the glycoprotein into GP1 and GP2 subunits that remain associated stably in the prefusion conformation state.

The term “prevent”, “preventing” and “prevention” refers to the inhibition of the development or onset of a condition (e.g., a Lassa virus infection or a condition associated therewith), or the prevention of the recurrence, onset, or development of one or more symptoms of a condition in a subject resulting from the administration of a therapy or the administration of a combination of therapies.

The term “prophylactically effective amount” refers to the amount of a composition (e.g., the recombinant MVA vector or pharmaceutical composition) which is sufficient to result in the prevention of the development, recurrence, or onset of a condition or a symptom thereof (e.g., an ebolavirus infection or a condition or symptom associated therewith or to enhance or improve the prophylactic effect(s) of another therapy.

The term “recombinant” means a polynucleotide of semisynthetic, or synthetic origin that either does not occur in nature or is linked to another polynucleotide in an arrangement not found in nature.

The term “recombinant,” with respect to a viral vector, means a vector (e.g., a viral genome that has been manipulated in vitro, e.g., using recombinant nucleic acid techniques to express heterologous viral nucleic acid sequences.

The term “regulatory sequence” “regulatory sequences” refers collectively to promoter sequences, polyadenylation signals, transcription termination sequences, upstream regulatory domains, origins of replication, internal ribosome entry sites (“IRES”), enhancers, and the like, which collectively provide for the transcription and translation of a coding sequence. Not all of these control sequences need always be present so long as the selected gene is capable of being transcribed and translated.

The term “shuttle vector” refers to a genetic vector (e.g., a DNA plasmid) that is useful for transferring genetic material from one host system into another. A shuttle vector can replicate alone (without the presence of any other vector) in at least one host (e.g., E. coli). In the context of MVA vector construction, shuttle vectors are usually DNA plasmids that can be manipulated in E. coli and then introduced into cultured cells infected with MVA vectors, resulting in the generation of new recombinant MVA vectors.

The term “silent mutation” means a change in a nucleotide sequence that does not cause a change in the primary structure of the protein encoded by the nucleotide sequence, e.g., a change from AAA (encoding lysine) to AAG (also encoding lysine).

The term “subject” is means any mammal, including but not limited to, humans, domestic and farm animals, and zoo, sports, or pet animals, such as dogs, horses, cats, cows, rats, mice, guinea pigs and the like.

The term “surrogate endpoint” means a clinical measurement other than a measurement of clinical benefit that is used as a substitute for a measurement of clinical benefit.

The term “surrogate marker” means a laboratory measurement or physical sign that is used in a clinical or animal trial as a substitute for a clinically meaningful endpoint that is a direct measure of how a subject feels, functions, or survives and is expected to predict the effect of the therapy (Katz, R., NeuroRx 1:189-195 (2004); New drug, antibiotic, and biological drug product regulations; accelerated approval—FDA. Final rule. Fed Regist 57: 58942-58960, 1992.)

The term “surrogate marker for protection” means a surrogate marker that is used in a clinical or animal trial as a substitute for the clinically meaningful endpoint of prevention of ebolavirus or marburgvirus infection.

The term “synonymous codon” refers to the use of a codon with a different nucleic acid sequence to encode the same amino acid, e.g., AAA and AAG (both of which encode lysine). Codon optimization changes the codons for a protein to the synonymous codons that are most frequently used by a vector or a host cell.

The term “therapeutically effective amount” means the amount of the composition (e.g., the recombinant MVA vector or pharmaceutical composition) that, when administered to a mammal for treating an infection, is sufficient to effect such treatment for the infection.

The term “treating” or “treat” refer to the eradication or control of a Lassa virus, a reduction in the titer of the Lassa virus, a reduction in the numbers of the Lassa virus, the reduction or amelioration of the progression, severity, and/or duration of a condition or one or more symptoms caused by the Lassa virus resulting from the administration of one or more therapies, or the reduction or elimination of the subject's ability to transmit the infection to another, uninfected subject.

The term “vaccine” means material used to provoke an immune response and confer immunity after administration of the material to a subject. Such immunity may include a cellular or humoral immune response that occurs when the subject is exposed to the immunogen after vaccine administration.

The term “vaccine insert” refers to a nucleic acid sequence encoding a heterologous sequence that is operably linked to a promoter for expression when inserted into a recombinant vector. The heterologous sequence may encode a glycoprotein or matrix protein described here.

The term “viral infection” means an infection by a viral pathogen (e.g., a member of genus Ebolavirus) wherein there is clinical evidence of the infection based on symptoms or based on the demonstration of the presence of the viral pathogen in a biological sample from the subject.

The term “virus-like particles” or “VLP” refers to a structure which resembles the native virus antigenically and morphologically.

The term “VP40” refers to a virus large matrix protein, or the gene or transcript encoding a virus large matrix protein.

### II. Lassa Virus Species and Sequences

Lassa virus is an arenavirus belonging to genus Arenavirus, family Arenaviridae. The arenavirus genome consists of two single-stranded negative-sense RNAs, one approximately 7.2 kb in length and the other approximately 3.5 kb in length each containing sequences encoding the matrix (Z) protein and glycoprotein (GP) respectively. As the sole antigen on the virus surface, the glycoprotein complex is the primary target for protective humoral immune responses and for vaccine design.

A study of more than 100 survivors of Lassa virus infection showed that the majority of neutralizing responses to Lassa virus bound to the quaternary assembly of the prefusion glycoprotein complex trimer rather than either subunit alone (Robinson, J. E., et al. Nature Commun. 7, 11544, 2016). A previous study of the 3.2 Angstrom crystal structure of the prefusion glycoprotein complex trimer of Lassa virus in complex with human neutralizing antibody 37.7H showed that conformational changes occur in the GP1 and GP2 glycoprotein subunits upon exposure to low pH. While not to be bound by any theory, it is believed that these changes suggest that the glycoprotein complex must be enzymatically processed to oligomerize and bind extracelluluar receptors and more importantly that neutralizing antibodies function by blocking the conformational changes that are required for binding an intracellular receptor and fusion. (Hastie, K. M., et al., Science, 256, 923-928, 2017)

Lassa fever is the acute hemorrhagic fever caused by Lassa virus. Symptoms typically appear 6-21 days after infection. Approximately 80% of cases are mild, involving mild fever, general malaise, weakness, and headache. In approximately 20% of cases, Lassa fever causes more severe symptoms including high fever, sore throat, mucosal bleeding, respiratory distress, vomiting, swelling, severe pain, and shock. Certain neurological problems may also occur. Of patients hospitalized for Lassa fever, approximately 15%-20% die from the infection (Kyei et al. (2015), BMC Infectious Diseases 15:217). Unlike filoviruses, which cause sporadic outbreaks, Lassa virus is a common human pathogen that causes endemic disease in a large area of West Africa (Andersen et al. (2015), Cell 162:738-750). Official estimates indicate 300,000-500,000 cases of Lassa fever each year with approximately 5,000-10,000 deaths; however, other measures indicate that the disease may be much more serious, accounting for as many as 3 million cases and 67,000 deaths annually (Leski et al. (2015) Emerging Infectious Diseases 21(4):609-618). Several experimental vaccines against LASV have been tested in animal models. To date, however, no Lassa fever vaccine has yet been approved for sale (Falzarano and Feldmann (2015), Current Opinion in Virology 3:343-351). Other than supportive care, there are few options for treatment of Lassa virus infection. Only the broad-spectrum antiviral drug ribavirin has shown efficacy, and it must be used early in the course of the disease in order to be effective (Ölschläger and Flatz (2013), PLoS Pathogens 9(4):e1003212).

A modified Lassa virus glycoprotein binds to human neutralizing antibody 37.7H. The structure of Lassa virus glycoprotein ectodomain may be modified using some or all of the following modifications: (a) point mutations R207C and G360C to covalently link GP1 and GP2 together, (b) introduce a proline via an E329P mutation in the metastable region of HR1 of RP2 and/or (c) replacing the native S1P GP1-GP2 cleavage site (RRLL at approximately position 277 of SEQ ID NO:2) with a furin site (i.e. RRLL to RRRR) to enable efficient processing the GP. Size-exclusion chromatography and multiangle light scattering (SEC-MALS) and SDS-polyacrylamide-gel electrophoresis analysis demonstrates that the Lassa glycoprotein is expressed as a single unit, and that the protein is efficiently processed into GP1 and GP2 subunits that remain associated. The GPCysR4 is recognized by neutralizing antibodies that required native association between the GP1 and GP2 subunits demonstrating a prefusion form of Lassa virus glycoprotein. Neutralizing antibody 37.7H against Lassa virus was isolated from a Sierra Leone survivor of Lassa fever. The 37.7H antibody neutralizes viruses representing all four known lineages of Lassa virus in vitro and in guinea pig challenges. (Robinson, J. E., et al. Nature Commun. 7, 11544, 2016; Cross R. W. et al., Antiviral Res. 133, 218-222, 2016). While not to be bound by any particular theory, it is believed that the 37.7H antibody neutralizes the virus by stabilizing glycoprotein complex in the prefusion conformation, thereby preventing the conformational changes required for Lassa virus infection. Another antibody 12.1F is known to bind to the upper beta sheet face of Lassa virus GP1 and is presumed to block cell attachment. (Robinson, J. E., et al. Nature Commun. 7, 11544, 2016).

In various embodiments, the following combinations of modifications are included in MVA vectors.

TABLE 1
MVA Viral Vector Sequences

| | Glycoprotein (GP) | GP Nucleic Acid | GP Protein | Z protein |
|---|---|---|---|---|
| | sequence mutations | Sequence | Sequence | (nucleic acid/protein sequence) |
| 1 | wild type | SEQ ID NO: 1 | SEQ ID NO: 2 | SEQ ID NO: 17/SEQ ID NO: 18 |
| 2 | N99D | SEQ ID NO: 3 | SEQ ID NO: 4 | SEQ ID NO: 17/SEQ ID NO: 18 |
| 3 | N119D | SEQ ID NO: 5 | SEQ ID NO: 6 | SEQ ID NO: 17/SEQ ID NO: 18 |
| 4 | N99D and N119D | SEQ ID NO: 7 | SEQ ID NO: 8 | SEQ ID NO: 17/SEQ ID NO: 18 |
| 5 | R207C, E329P, and G360C | SEQ ID NO: 9 | SEQ ID NO: 10 | SEQ ID NO: 17/SEQ ID NO: 18 |
| 6 | R207C, E329P, G360C and | SEQ ID NO: 11 | SEQ ID NO: 12 | SEQ ID NO: 17/SEQ ID NO: 18 |
| | N99D | | | |
| 7 | R207C, E329P, G360C and | SEQ ID NO: 13 | SEQ ID NO: 14 | SEQ ID NO: 17/SEQ ID NO: 18 |
| | N119D | | | |
| 8 | R207C, E329P, G360C, | SEQ ID NO: 15 | SEQ ID NO: 16 | SEQ ID NO: 17/SEQ ID NO: 18 |
| | N99D and N119D | | | |

In one embodiment, a Lassa virus deglycosylation mutant glycoprotein is expressed by a recombinant viral vector to induce an immune response to Lassa virus.

In one embodiment, a prefusion Lassa virus glycoprotein is expressed by a recombinant viral vector to induce an immune response to Lassa virus.

In one embodiment, the prefusion Lassa virus glycoprotein is encoded by SEQ ID NO:9 and expresses as SEQ ID NO:10. (See Hastie et al., Science 356, 923-928 (2017).

In one embodiment, the expressed prefusion Lassa virus glycoprotein binds to human neutralizing antibody 37.7H and/or 12.1F.

### III. Recombinant Viral Vectors

In one aspect, the present invention is a recombinant viral vector comprising one or more genes of a Lassa virus. In certain embodiments, the recombinant viral vector is a vaccinia viral vector, and more particularly, an MVA vector, comprising one or more genes of a Lassa virus.

Vaccinia viruses have also been used to engineer viral vectors for recombinant gene expression and for the potential use as recombinant live vaccines (Mackett, M. et al 1982 PNAS USA 79:7415-7419; Smith, G. L. et al. 1984 Biotech Genet Engin Rev 2:383-407). This entails DNA sequences (genes) which code for foreign antigens being introduced, with the aid of DNA recombination techniques, into the genome of the vaccinia viruses. If the gene is integrated at a site in the viral DNA which is non-essential for the life cycle of the virus, it is possible for the newly produced recombinant vaccinia virus to be infectious, that is to say able to infect foreign cells and thus to express the integrated DNA sequence (EP Patent Applications No. 83,286 and No. 110,385). The recombinant vaccinia viruses prepared in this way can be used, on the one hand, as live vaccines for the prophylaxis of infectious diseases, on the other hand, for the preparation of heterologous proteins in eukaryotic cells.

Several such strains of vaccinia virus have been developed to avoid undesired side effects of smallpox vaccination. Thus, a modified vaccinia Ankara (MVA) has been generated by long-term serial passages of the Ankara strain of vaccinia virus (CVA) on chicken embryo fibroblasts (for review see Mayr, A. et al. 1975 Infection 3:6-14; Swiss Patent No. 568,392). The MVA virus is publicly available from American Type Culture Collection as ATCC No.: VR-1508. MVA is distinguished by its great attenuation, as demonstrated by diminished virulence and reduced ability to replicate in primate cells, while maintaining good immunogenicity. The MVA virus has been analyzed to determine alterations in the genome relative to the parental CVA strain. Six major deletions of genomic DNA (deletion I, II, III, IV, V, and VI) totaling 31,000 base pairs have been identified (Meyer, H. et al. 1991 J Gen Virol 72:1031-1038). The resulting MVA virus became severely host cell restricted to avian cells.

Furthermore, MVA is characterized by its extreme attenuation. When tested in a variety of animal models, MVA was proven to be avirulent even in immunosuppressed animals. More importantly, the excellent properties of the MVA strain have been demonstrated in extensive clinical trials (Mayr A. et al. 1978 Zentralbl Bakteriol [B] 167:375-390; Stickl et al. 1974 Dtsch Med Wschr 99:2386-2392). During these studies in over 120,000 humans, including high-risk patients, no side effects were associated with the use of MVA vaccine.

MVA replication in human cells was found to be blocked late in infection preventing the assembly to mature infectious virions. Nevertheless, MVA was able to express viral and recombinant genes at high levels even in non-permissive cells and was proposed to serve as an efficient and exceptionally safe gene expression vector (Sutter, G. and Moss, B. 1992 PNAS USA 89:10847-10851). Additionally, novel vaccinia vector vaccines were established on the basis of MVA having foreign DNA sequences inserted at the site of deletion III within the MVA genome (Sutter, G. et al. 1994 Vaccine 12:1032-1040).

Recombinant MVA vaccinia viruses can be prepared as set out hereinafter. A DNA-construct which contains a DNA-sequence which codes for a foreign polypeptide flanked by MVA DNA sequences adjacent to a predetermined insertion site (e.g. between two conserved essential MVA genes such as I8R/G1L; in restructured and modified deletion III; or at other non-essential sites within the MVA genome) is introduced into cells infected with MVA, to allow homologous recombination. Once the DNA-construct has been introduced into the eukaryotic cell and the foreign DNA has recombined with the viral DNA, it is possible to isolate the desired recombinant vaccinia virus in a manner known per se, preferably with the aid of a marker. The DNA-construct to be inserted can be linear or circular. A plasmid or polymerase chain reaction product is preferred. Such methods of making recombinant MVA vectors are described in PCT publication WO/2006/026667 incorporated by reference herein. The DNA-construct contains sequences flanking the left and the right side of a naturally occurring deletion. The foreign DNA sequence is inserted between the sequences flanking the naturally occurring deletion. For the expression of a DNA sequence or gene, it is necessary for regulatory sequences, which are required for the transcription of the gene, to be present on the DNA. Such regulatory sequences (called promoters) are known to those skilled in the art, and include for example those of the vaccinia 11 kDa gene as are described in EP-A-198,328, and those of the 7.5 kDa gene (EP-A-110,385). The DNA-construct can be introduced into the MVA infected cells by transfection, for example by means of calcium phosphate precipitation (Graham et al. 1973 Virol 52:456-467; Wigler et al. 1979 Cell 16:777-785), by means of electroporation (Neumann et al. 1982 EMBO J. 1:841-845), by microinjection (Graessmann et al. 1983 Meth Enzymol 101:482-492), by means of liposomes (Straubinger et al. 1983 Meth Enzymol 101:512-527), by means of spheroplasts (Schaffher 1980 PNAS USA 77:2163-2167) or by other methods known to those skilled in the art.

The MVA-VLP platform has multiple advantages over other live attenuated or replicating vectors that can have significant reactogenicity and inherent safety concerns for use in immunocompromised individuals (e.g. HIV or cancer patients), infants, and women of child-bearing age. The MVA is replication deficient in humans; it has inherent and proven safety in immunocompromised individuals including HIV patients. Moreover, the mutated or stabilized GPCs will likely render the protein non-functional (incapable of binding to its cellular receptor or undergoing conformational changes necessary for endosomal fusion and infectivity), they cannot be easily used in replicating vectors that require a functional GPC on their surface (e.g. VSV-LASV). In contrast, the MVA vectors described herein use their own fusion machinery and do not require a functional LASV GPC for infectivity in avian or mammalian cells.

The MVA vectors described and tested herein were unexpectedly found to be effective after a single prime or a homologous prime/boost regimen. Other MVA vector designs require a heterologous prime/boost regimen while still other published studies have been unable to induce effective immune responses with MVA vectors. Conversely, the present MVA vector design and methods of manufacture are useful in producing effective MVA vaccine vectors for eliciting effective T-cell and antibody immune responses. Furthermore, the utility of an MVA vaccine vector capable of eliciting effective immune responses and antibody production after a single homologous prime boost is significant for considerations such as use, commercialization and transport of materials especially to affected third world locations.

In one embodiment, the present invention is a recombinant viral vector (e.g., an MVA vector) comprising one or more heterologous gene inserts of a filovirus (e.g., an ebolavirus or marburgvirus). The viral vector (e.g., an MVA vector) may be constructed using conventional techniques known to one of skill in the art. The one or more heterologous gene inserts encode a polypeptide having desired immunogenicity, i.e., a polypeptide that can induce an immune reaction, cellular immunity and/or humoral immunity, in vivo by administration thereof. The gene region of the viral vector (e.g., an MVA vector) where the gene encoding a polypeptide having immunogenicity is introduced is flanked by regions that are indispensable. In the introduction of a gene encoding a polypeptide having immunogenicity, an appropriate promoter may be operatively linked upstream of the gene encoding a polypeptide having desired immunogenicity.

In various embodiments, the recombinant viral vector comprises one or more genes encoding a Lassa virus prefusion glycoprotein, and a viral matrix protein. In exemplary embodiments, the gene encodes a polypeptide or protein capable of inducing an immune response in the subject to which it is administered, and more particularly, an immune response capable of providing a protective and/or therapeutic benefit to the subject. In one embodiment, the one or more genes encode a Lassa virus prefusion glycoprotein (GP), and/or one or more viral matrix proteins (e.g., Z, VP40, VP35, VP30, or VP24). The heterologous gene inserts are inserted into one or more deletion sites of the vector under the control of promoters compatible with poxvirus expression systems.

In one embodiment, the deletion III site is restructured and modified to remove non-essential flanking sequences.

In exemplary embodiments, the vaccine is constructed to express a Lassa virus glycoprotein, where an encoding sequence is inserted between two conserved essential MVA genes (for example I8R and G1L) using shuttle vector pGeo-LASV_GP; and to express Lassa virus Z protein, which is inserted into deletion III using shuttle vector pGeo-LASV_Z. pGeo-LASV_GP and pGeo-LASV_Z are constructed with an ampicillin resistance marker, allowing the vector to replicate in bacteria; with two flanking sequences, allowing the vector to recombine with a specific location in the MVA genome; with a green fluorescent protein (GFP) selection marker, allowing the selection of recombinant MVAs; with a sequence homologous to part of Flank 1 of the MVA sequence, enabling removal of the GFP sequence from the MVA vector after insertion of VP40 into the MVA genome; with a modified H5 (mH5) promoter, which enables transcription of the inserted heterologous gene insert; and with a arenavirus gene. pGeo-GP and pGeo-LasZ differ in that pGeo-GP contains the GP sequence, whereas pGeo-LasZ contains the Lassa virus Z sequence; and in that pGeo-GP recombines with sequences of MVA I8R and G1L (two essential genes) and pGeo-LasZ recombines with regions flanking the restructured and modified Deletion III of MVA.

In exemplary embodiments, the present invention provides a recombinant MVA vector comprising a gene encoding a Lassa virus glycoprotein (GP) gene and a gene encoding Lassa virus Z protein.

In exemplary embodiments, the present invention provides a recombinant MVA vector comprising a gene encoding a Lassa virus glycoprotein (GP) gene and a gene encoding VP40, selected from an ebolavirus, or marburgvirus.

In certain embodiments, the polypeptide, or the nucleic acid sequence encoding the polypeptide, may have a mutation or deletion (e.g., an internal deletion, truncation of the amino- or carboxy-terminus, or a point mutation).

The one or more genes introduced into the recombinant viral vector are under the control of regulatory sequences that direct its expression in a cell.

The nucleic acid material of the viral vector may be encapsulated, e.g., in a lipid membrane or by structural proteins (e.g., capsid proteins), that may include one or more viral polypeptides.

In exemplary embodiments, the present invention is a recombinant viral vector (e.g., a recombinant MVA vector) comprising one or more genes, or one or more polypeptides encoded by the gene or genes, from a Lassa virus. The Lassa virus gene may encode a polypeptide or protein capable of inducing an immune response in the subject to which it is administered, and more particularly, an immune response capable of providing a protective and/or therapeutic benefit to the subject, e.g., the Lassa virus prefusion glycoprotein. The nucleic acid sequences of Lassa virus glycoprotein and matrix proteins are published and are available from a variety of sources, including, e.g., GenBank and PubMed. Exemplary GenBank references including Lassa virus glycoprotein and matrix sequences include those corresponding to accession numbers JN650517 (LASV GP and NP) and JN650518 (LASV Z).

In certain embodiments, the one or more genes encodes a polypeptide, or fragment thereof, that is substantially identical (e.g., at least 60%, 65%, 70%, 75%, 80%, 85%, 90%, 95%, 98%, 99%, or even 100% identical) to the selected Lassa virus glycoprotein over at least 20, 25, 30, 35, 40, 45, 50, 55, 60, 65, or 70 contiguous residues of the selected Lassa virus glycoprotein that retain immunogenic activity.

In exemplary embodiments, the recombinant viral vector may also include an Lassa virus glycoprotein present on its surface. The Lassa virus glycoprotein may be obtained by any suitable means, including, e.g., application of genetic engineering techniques to a viral source, chemical synthesis techniques, recombinant production, or any combination thereof.

In another embodiments, the present invention is a recombinant MVA vector comprising at least one heterologous gene insert from a Lassa virus, wherein the gene is selected from the group encoding the glycoprotein (GP), the secreted GP (sGP), the major nucleoprotein (NP), RNA-dependent RNA polymerase (L), or one or more other viral proteins (e.g., Z, VP40, VP35, VP30, or VP24).

In a particular embodiment, the present invention is a recombinant MVA vector comprising a gene encoding a Lassa virus glycoprotein selected from either a stabilized prefusion glycoprotein or deglycosylation mutant glycoprotein sequence and a gene encoding a matrix protein such as Z protein or VP40. In another embodiment, the present invention is a recombinant MVA vector comprising genes encoding Lassa virus glycoprotein selected from either a stabilized prefusion glycoprotein or deglycosylation mutant glycoprotein sequence, Z, and NP. The heterologous gene inserts are inserted into one or more deletion sites of the MVA vector under the control of promoters compatible with poxvirus expression systems.

In one embodiment, the Lassa virus glycoprotein is inserted into deletion site I, II, III, IV, V or VI of the MVA vector, and the VP40 is inserted into deletion site I, II, III, IV, V or VI of the MVA vector.

In one embodiment, the Lassa virus glycoprotein is inserted between I8R and G1 L of the MVA vector, or into restructured and modified deletion III of the MVA vector; and the VP40 is inserted between I8R and G1L of the MVA vector, or into restructured and modified deletion site III of the MVA vector.

In one embodiment relating to LASV, the Lassa virus glycoprotein is inserted into deletion site I, II, III, IV, V or VI of the MVA vector, and the Z is inserted into deletion site I, II, III, IV, V or VI of the MVA vector.

In one embodiment, the recombinant vector comprises in a first deletion site, a gene encoding Lassa virus glycoprotein operably linked to a promoter compatible with poxvirus expression systems, and in a second deletion site, genes encoding Z and NP in reverse orientation each operably linked to a promoter compatible with poxvirus expression systems.

In one embodiment relating to LASV, the Lassa virus glycoprotein is inserted between I8R and G1 L of the MVA vector, or into restructured and modified deletion III of the MVA vector; and the Z is inserted between I8R and G1L of the MVA vector, or into restructured and modified deletion site III of the MVA vector.

In another embodiment relating to LASV, the Lassa virus glycoprotein and Z are inserted into different deletion sites. For example, the GP sequence is inserted between two essential and highly conserved MVA genes, I8R/G1L, to limit the formation of viable deletion mutants; and, the Z sequence is inserted into a restructured and modified deletion III site.

In exemplary embodiments, the present invention is a recombinant MVA vector comprising at least one heterologous gene insert (e.g., one or more gene inserts) from an ebolavirus or a marburgvirus which is under the control of regulatory sequences that direct its expression in a cell. The gene may be, for example, under the control of a promoter selected from the group consisting of Pm2H5, Psyn II, or mH5 promoters.

The recombinant viral vector of the present invention can be used to infect cells of a subject, which, in turn, promotes the translation into a protein product of the one or more viral genes of the viral vector (e.g., Lassa virus glycoprotein). As discussed further herein, the recombinant viral vector can be administered to a subject so that it infects one or more cells of the subject, which then promotes expression of the one or more viral genes of the viral vector and stimulates an immune response that is protective against infection by a Lassa virus, or that reduces or prevents infection by a Lassa virus.

In one embodiment, the recombinant MVA vaccine expresses proteins that assemble into virus-like particles (VLPs) comprising the Lassa virus prefusion glycoprotein, and VP40 (matrix protein). While not wanting to be bound by any particular theory, it is believed that the Lassa virus prefusion glycoprotein is provided to elicit a protective immune response and the VP40 (matrix protein) is provided to enable assembly of VLPs and as a target for T cell immune responses, thereby enhancing the protective immune response and providing cross-protection.

Similarly relating to LASV, in one embodiment, the recombinant MVA vaccine expresses proteins that assemble into virus-like particles (VLPs) comprising the Lassa virus prefusion glycoprotein (glycoprotein), and Z (matrix protein). While not wanting to be bound by any particular theory, it is believed that the Lassa virus prefusion glycoprotein is provided to elicit a protective immune response and the Z (matrix protein) is provided to enable assembly of VLPs and as a target for T cell immune responses, thereby enhancing the protective immune response and providing cross-protection (i.e. antibody and T cell responses).

For references, see Stahelin, Front in Microbiol 5:300 (2014); Marzi et al., J Infect Dis 204 Suppl 3:S1066 (2011); Warfield and Aman, J Infect Dis 204 Suppl 3:S1053 (2011); and Mire et al., PLoS Negl Trop Dis 7:e2600 (2013).

One or more genes may be optimized for use in an MVA vector. Optimization includes codon optimization, which employs silent mutations to change selected codons from the native sequences into synonymous codons that are optimally expressed by the host-vector system. Other types of optimization include the use of silent mutations to interrupt homopolymer stretches or transcription terminator motifs. Each of these optimization strategies can improve the stability of the gene, improve the stability of the transcript, or improve the level of protein expression from the gene. In exemplary embodiments, the number of homopolymer stretches in the Lassa virus prefusion glycoprotein or VP40 sequence will be reduced to stabilize the construct.

In exemplary embodiments, the Lassa virus glycoprotein and matrix protein sequences are codon optimized for expression in MVA using a computer algorithm; Lassa virus prefusion glycoprotein and VP40 sequences with runs of ≥5 deoxyguanosines, ≥5 deoxycytidines, ≥5 deoxyadenosines, and ≥5 deoxythymidines are interrupted by silent mutation to minimize loss of expression due to frame shift mutations; and the Lassa virus prefusion glycoprotein sequence is modified through addition of an extra nucleotide to express the transmembrane, rather than the secreted, form of Lassa virus prefusion glycoprotein.

In one embodiment, the present invention provides a vaccine vector composition that is monovalent. As used herein the term monovalent refers to a vaccine vector composition that contains Lassa virus prefusion glycoprotein and matrix sequences from one species of Ebolavirus, Marbugvirus, or Arenavirus.

In another embodiment, the present invention provides a vaccine that is bivalent. As used herein the term bivalent refers to a vaccine vector composition that contains two vectors each having a sequence encoding a Lassa virus glycoprotein and Lassa virus prefusion glycoprotein, and each having a sequence encoding a matrix protein from the same or different species of Ebolavirus, Marbugvirus, or Arenavirus.

In another embodiment, the present invention provides a vaccine that is trivalent. As used herein the term trivalent refers to a vaccine vector composition that contains three vectors each having a sequence expressing a Lassa virus glycoprotein, wherein one vector has a sequence expressing a Lassa virus prefusion glycoprotein and each having a sequence encoding a matrix sequence from the same or different species of Ebolavirus, Marbugvirus, or Arenavirus.

The recombinant viral vectors of the present invention may be used alone, or in combination. In one embodiment, two different recombinant viral vectors are used in combination, where the difference may refer to the one or more heterologous gene inserts or the other components of the recombinant viral vector or both. In exemplary embodiments, two or more recombinant viral vectors are used in combination in order to protect against infection by all forms of Lassa virus known to be lethal in humans.

The present invention also extends to host cells comprising the recombinant viral vector described above, as well as isolated virions prepared from host cells infected with the recombinant viral vector.

### IV. Pharmaceutical Composition

The recombinant viral vectors of the present invention are readily formulated as pharmaceutical compositions for veterinary or human use, either alone or in combination. The pharmaceutical composition may comprise a pharmaceutically acceptable diluent, excipient, carrier, or adjuvant.

In one embodiment, the present invention is a vaccine effective to protect and/or treat a Lassa virus comprising a recombinant MVA vector that expresses at least one Lassa virus prefusion glycoprotein or an immunogenic fragment thereof. The vaccine composition may comprise one or more additional therapeutic agents.

The pharmaceutical composition may comprise 1, 2, 3, 4 or more than 4 different recombinant MVA vectors.

In one embodiment, the present invention provides a vaccine vector composition that is monovalent. As used herein the term monovalent refers to a vaccine vector composition that contains Lassa virus prefusion glycoprotein and matrix sequences from one species of Ebolavirus, Marbugvirus, or Arenavirus.

In another embodiment, the present invention provides a vaccine that is bivalent. As used herein the term bivalent refers to a vaccine vector composition that contains two vectors each having a sequence encoding a Lassa virus glycoprotein and Lassa virus prefusion glycoprotein, and each having a sequence encoding a matrix protein from the same or different species of Ebolavirus, Marbugvirus, or Arenavirus.

As used herein, the phrase “pharmaceutically acceptable carrier” encompasses any of the standard pharmaceutical carriers, such as those suitable for parenteral administration, such as, for example, by intramuscular, intraarticular (in the joints), intravenous, intradermal, intraperitoneal, and subcutaneous routes. Examples of such formulations include aqueous and non-aqueous, isotonic sterile injection solutions, which contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. One exemplary pharmaceutically acceptable carrier is physiological saline.

Other physiologically acceptable diluents, excipients, carriers, or adjuvants and their formulations are known to those skilled in the art.

The compositions utilized in the methods described herein can be administered by a route selected from, e.g., parenteral, intramuscular, intraarterial, intravascular, intravenous, intraperitoneal, subcutaneous, dermal, transdermal, ocular, inhalation, buccal, sublingual, perilingual, nasal, topical administration, and oral administration. The preferred method of administration can vary depending on various factors (e.g., the components of the composition being administered, and the severity of the condition being treated). Formulations suitable for oral administration may consist of liquid solutions, such as an effective amount of the composition dissolved in a diluent (e.g., water, saline, or PEG-400), capsules, sachets or tablets, each containing a predetermined amount of the vaccine. The pharmaceutical composition may also be an aerosol formulation for inhalation, e.g., to the bronchial passageways. Aerosol formulations may be mixed with pressurized, pharmaceutically acceptable propellants (e.g., dichlorodifluoromethane, propane, or nitrogen).

For the purposes of this invention, pharmaceutical compositions suitable for delivering a therapeutic or biologically active agent can include, e.g., tablets, gelcaps, capsules, pills, powders, granulates, suspensions, emulsions, liposomes, solutions, gels, hydrogels, oral gels, pastes, eye drops, ointments, creams, plasters, drenches, delivery devices (e.g. needle free injections, miconeedle patches, and solid formulation delivery by needle free devices), suppositories, enemas, injectables, implants, sprays, or aerosols. Any of these formulations can be prepared by well-known and accepted methods of art. See, for example, Remington: The Science and Practice of Pharmacy (21.sup.st ed.), ed. A. R. Gennaro, Lippincott Williams & Wilkins, 2005, and Encyclopedia of Pharmaceutical Technology, ed. J. Swarbrick, Informa Healthcare, 2006, each of which is hereby incorporated by reference.

The immunogenicity of the composition (e.g., vaccine) may be significantly improved if the composition of the present invention is co-administered with an immunostimulatory agent or adjuvant. Suitable adjuvants well-known to those skilled in the art include, e.g., aluminum phosphate, aluminum hydroxide, QS21, Quil A (and derivatives and components thereof), calcium phosphate, calcium hydroxide, zinc hydroxide, glycolipid analogs, octodecyl esters of an amino acid, muramyl dipeptides, polyphosphazene, lipoproteins, ISCOM-Matrix, DC-Chol, DDA, cytokines, and other adjuvants and derivatives thereof.

Pharmaceutical compositions according to the invention described herein may be formulated to release the composition immediately upon administration (e.g., targeted delivery) or at any predetermined time period after administration using controlled or extended release formulations. Administration of the pharmaceutical composition in controlled or extended release formulations is useful where the composition, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀); (ii) a narrow absorption window in the gastro-intestinal tract; or (iii) a short biological half-life, so that frequent dosing during a day is required in order to sustain a therapeutic level.

Many strategies can be pursued to obtain controlled or extended release in which the rate of release outweighs the rate of metabolism of the pharmaceutical composition. For example, controlled release can be obtained by the appropriate selection of formulation parameters and ingredients, including, e.g., appropriate controlled release compositions and coatings. Suitable formulations are known to those of skill in the art. Examples include single or multiple unit tablet or capsule compositions, oil solutions, suspensions, emulsions, microcapsules, microspheres, nanoparticles, patches, and liposomes.

Formulations suitable for oral administration can consist of (a) liquid solutions, such as an effective amount of the vaccine dissolved in diluents, such as water, saline or PEG 400; (b) capsules, sachets or tablets, each containing a predetermined amount of the vaccine, as liquids, solids, granules or gelatin; (c) suspensions in an appropriate liquid; (d) suitable emulsions; and (e) polysaccharide polymers such as chitins. The vaccine, alone or in combination with other suitable components, may also be made into aerosol formulations to be administered via inhalation, e.g., to the bronchial passageways. Aerosol formulations can be placed into pressurized acceptable propellants, such as dichlorodifluoromethane, propane, nitrogen, and the like.

Suitable formulations for rectal administration include, for example, suppositories, which consist of the vaccine with a suppository base. Suitable suppository bases include natural or synthetic triglycerides or paraffin hydrocarbons. In addition, it is also possible to use gelatin rectal capsules which consist of a combination of the vaccine with a base, including, for example, liquid triglycerides, polyethylene glycols, and paraffin hydrocarbons.

Pharmaceutical compositions comprising any of the nucleic acid molecules encoding Lassa viral proteins of the present invention are useful to immunize a subject against disease caused by Lassa infection. Thus, this invention further provides methods of immunizing a subject against disease caused by Lassa virus infection, e.g., Lassa fever, comprising administering to the subject an immunoeffective amount of a pharmaceutical composition of the invention. This subject may be an animal, for example a mammal, such as a primate or preferably a human.

The vaccines of the present invention may also be co-administered with cytokines to further enhance immunogenicity. The cytokines may be administered by methods known to those skilled in the art, e.g., as a nucleic acid molecule in plasmid form or as a protein or fusion protein.

### Kits

This invention also provides kits comprising the vaccines of the present invention. For example, kits comprising a vaccine and instructions for use are within the scope of this invention.

### V. Method of Use

The compositions of the invention can be used as vaccines for inducing an immune response to an arenavirus, such Lassa virus including any species thereof.

In exemplary embodiments, the present invention provides a method of preventing an arenavirus (e.g., Lassa virus) infection to a subject in need thereof (e.g., an unexposed) subject, said method comprising administering the composition of the present invention to the subject in a prophylactically effective amount. The result of the method is that the subject is partially or completely immunized against the virus.

In exemplary embodiments, the present invention provides a method of treating a arenavirus (e.g., Lassa virus) infection in a subject in need thereof (e.g., an exposed subject, such as a subject who has been recently exposed but is not yet symptomatic, or a subject who has been recently exposed and is only mildly symptomatic), said method comprising administering the composition of the present invention to the subject in a therapeutically effective amount. The result of treatment is a subject that has an improved therapeutic profile.

In certain embodiments, the compositions of the invention can be used as vaccines for treating a subject infected with more than one areavirus, e.g., multiple species of Arenavirus or various forms of arenavirus glycoprotein. The recombinant viral vector comprises genes or sequences encoding viral proteins of multiple species of Arenavirus and/or the pharmaceutical composition comprises more than one type of recombinant viral vector, in terms of the heterologous gene inserts or sequences contained.

Typically, the vaccines will be in an admixture and administered simultaneously but may also be administered separately.

A subject to be treated according to the methods described herein (e.g., a subject infected with, an ebolavirus) may be one who has been diagnosed by a medical practitioner as having such a condition. Diagnosis may be performed by any suitable means. A subject in whom the development of an infection is being prevented may or may not have received such a diagnosis. One skilled in the art will understand that a subject to be treated according to the present invention may have been identified using standard tests or may have been identified, without examination, as one at high risk due to the presence of one or more risk factors (e.g., exposure to ebolavirus, etc.).

Prophylactic treatment may be administered, for example, to a subject not yet exposed to or infected by a Lassa virus but who is susceptible to, or otherwise at risk of exposure or infection with a Lassa virus.

Therapeutic treatment may be administered, for example, to a subject already exposed to or infected by a hemorrhagic fever virus who is not yet ill, or showing symptoms or infection, suffering from a disorder in order to improve or stabilize the subject's condition (e.g., a patient already infected with a Lassa virus). The result is an improved therapeutic profile. In some instances, as compared with an equivalent untreated control, treatment may ameliorate a disorder or a symptom thereof by, e.g., 5%, 10%, 20%, 30%, 40%, 50%, 60%, 70%, 80%, 90%, 95%, or 100% as measured by any standard technique. In some instances, treating can result in the inhibition of viral replication, a decrease in viral titers or viral load, eradication or clearing of the virus.

In other embodiments, treatment may result in amelioration of one or more symptoms of the infection, including any symptom identified above. According to this embodiment, confirmation of treatment can be assessed by detecting an improvement in or the absence of symptoms.

In other embodiments, treatment may result in reduction or elimination of the ability of the subject to transmit the infection to another, uninfected subject. Confirmation of treatment according to this embodiment is generally assessed using the same methods used to determine amelioration of the disorder, but the reduction in viral titer or viral load necessary to prevent transmission may differ from the reduction in viral titer or viral load necessary to ameliorate the disorder.

In one embodiment, the present invention is a method of inducing an immune response in a subject (e.g., a human) by administering to the subject a recombinant viral vector that encodes at least one gene from a hemorrhagic fever virus, such as a member of genus Arenavirus. The immune response may be a cellular immune response or a humoral immune response, or a combination thereof.

In a particular embodiment, the present invention is a method of inducing an immune response in a subject (e.g., a human) by administering to the subject a recombinant viral vector that encodes at least one gene from a member of genus arenavirus. The immune response may be a cellular immune response or a humoral immune response, or a combination thereof.

In one embodiment, the immune response is a broadly neutralizing antibody response.

In a particular embodiment, the present invention is a method of inducing an immune response in a subject (e.g., a human) by administering to the subject a recombinant viral vector that encodes at least one gene from a member of genus Arenavirus, more particularly, LASV. In certain embodiments, the recombinant viral vector encodes at least two genes from an arenavirus, more particularly, LASV. The immune response may be a cellular immune response or a humoral immune response, or a combination thereof.

In another embodiment, the invention features a method of treating an arenavirus infection (e.g., a Lassa virus infection) in a subject (e.g., a human) by administering to the subject a recombinant viral vector that encodes at least one gene from the Lassa virus species of arenavirus (e.g., a LASV prefusion glycoprotein). The subject being treated may not have, but is at risk of developing, an infection by an arenavirus, for example, an infection caused by LASV.

In another embodiment, the subject may already be infected with at least one arenavirus (e.g., a Lassa virus).

The composition may be administered, e.g., by injection (e.g., intramuscular, intraarterial, intravascular, intravenous, intraperitoneal, or subcutaneous).

It will be appreciated that more than one route of administering the vaccines of the present invention may be employed either simultaneously or sequentially (e.g., boosting). In addition, the vaccines of the present invention may be employed in combination with traditional immunization approaches such as employing protein antigens, vaccinia virus and inactivated virus, as vaccines. Thus, in one embodiment, the vaccines of the present invention are administered to a subject (the subject is “primed” with a vaccine of the present invention) and then a traditional vaccine is administered (the subject is “boosted” with a traditional vaccine). In another embodiment, a traditional vaccine is first administered to the subject followed by administration of a vaccine of the present invention. In yet another embodiment, a traditional vaccine and a vaccine of the present invention are co-administered.

While not to be bound by any specific mechanism, it is believed that upon inoculation with a pharmaceutical composition as described herein, the immune system of the host responds to the vaccine by producing antibodies, both secretory and serum, specific for Lassa virus proteins; and by producing a cell-mediated immune response specific for Lassa virus. As a result of the vaccination, the host becomes at least partially or completely immune to Lassa virus infection, or resistant to developing moderate or severe disease caused by Lassa virus infection.

In one aspect, methods are provided to alleviate, reduce the severity of, or reduce the occurrence of, one or more of the symptoms (e.g., fever, severe headache, muscle pain, malaise, extreme asthenia, conjunctivitis, popular rash, dysphagia, nausea, vomiting, bloody diarrhea followed by diffuse hemorrhages, delirium, shock, jaundice, thrombocytopenia, lymphocytopenia, neutrophilia, focal necrosis in various organs (e.g., kidneys and liver), and acute respiratory distress) associated with Lassa virus infection comprising administering an effective amount of a pharmaceutical composition comprising a recombinant MVA viral vector that comprises a Lassa virus prefusion glycoprotein and VP40 sequences from the Zaire ebolavirus, Sudan ebolavirus, Taï Forest ebolavirus, Bundibugyo ebolavirus, Reston ebolavirus, or Marburg marburgvirus species of filovirus; or comprising GP and Z sequences from the Lassa virus species of arenavirus; or comprising GP, Z, and NP sequences from the Lassa virus species of arenavirus.

In one embodiment, the MVA viral vector comprises a prefusion glycoprotein and Z sequences from a Lassa virus species.

In one embodiment, the MVA viral vector comprises prefusion glycoprotein, Z, and NP sequences from a Lassa virus species.

In another aspect, the invention provides methods of inducing an immune response to Lassa virus comprising administering an effective amount of a pharmaceutical composition comprising a recombinant MVA vaccine expressing Lassa virus glycoprotein selected from either a stabilized prefusion glycoprotein or deglycosylation mutant glycoprotein sequence and matrix protein from at least one species of ebolavirus, marburgvirus, or Lassa virus. The Lassa vaccine of this aspect may also express the Lassa virus nucleoprotein.

In another aspect, the invention provides methods of providing anti-Lassa virus immunity comprising administering an effective amount of a pharmaceutical composition comprising a recombinant MVA vaccine expressing Lassa virus glycoprotein selected from either a stabilized prefusion glycoprotein or deglycosylation mutant glycoprotein sequence and matrix protein from at least one species of ebolavirus, marburgvirus, or Lassa virus. The Lassa vaccine of this aspect may also express the Lassa virus nucleoprotein.

In another aspect, the invention provides methods of reducing the spread of Lassa virus infection within a subject or from an infected subject to an uninfected subject, comprising administering an effective amount of a pharmaceutical composition comprising a recombinant MVA vaccine expressing Lassa virus glycoprotein selected from either a stabilized prefusion glycoprotein or deglycosylation mutant glycoprotein sequence and matrix protein from at least one species of ebolavirus, marburgvirus, or Lassa virus. The Lassa vaccine of this aspect may also express the Lassa virus nucleoprotein. In another aspect, the invention provides methods of reducing symptoms of Lassa virus infection comprising administering an effective amount of a pharmaceutical composition comprising a recombinant MVA vaccine expressing glycoprotein and matrix protein from at least one species of ebolavirus, marburgvirus, or Lassa virus. The Lassa vaccine of this aspect may also express the Lassa virus nucleoprotein. In another aspect, the invention provides methods of inducing an immune response which is considered a surrogate marker for protection against Lassa virus infection. Data for determination of whether a response constitutes a surrogate marker for protection are obtained using immune response data obtained using the measurements outlined above.

It will also be appreciated that single or multiple administrations of the vaccine compositions of the present invention may be carried out. For example, subjects who are particularly susceptible to Lassa virus infection may require multiple immunizations to establish and/or maintain protective immune responses. Levels of induced immunity can be monitored by measuring amounts of binding and neutralizing secretory and serum antibodies as well as levels of T cells, and dosages adjusted, or vaccinations repeated as necessary to maintain desired levels of protection.

In one embodiment, administration is repeated at least twice, at least 3 times, at least 4 times, at least 5 times, at least 6 times, at least 7 times, at least 8 times, or more than 8 times.

In one embodiment, administration is repeated twice.

In one embodiment, about 2-8, about 4-8, or about 6-8 administrations are provided.

In one embodiment, about 1-4-week, 2-4 week, 3-4 week, 1 week, 2 week, 3 week, 4 week or more than 4 week intervals are provided between administrations.

In one specific embodiment, a 4-week interval is used between 2 administrations.

### Dosage

The vaccines are administered in a manner compatible with the dosage formulation, and in such amount as will be therapeutically effective, immunogenic and protective. The quantity to be administered depends on the subject to be treated, including, for example, the capacity of the immune system of the individual to synthesize antibodies, and, if needed, to produce a cell-mediated immune response. Precise amounts of active ingredient required to be administered depend on the judgment of the practitioner and may be monitored on a patient-by-patient basis. However, suitable dosage ranges are readily determinable by one skilled in the art and generally range from about 5.0×10⁶ TCID₅₀ to about 5.0×10⁹ TCID₅₀. The dosage may also depend, without limitation, on the route of administration, the patient's state of health and weight, and the nature of the formulation.

The pharmaceutical compositions of the invention are administered in such an amount as will be therapeutically effective, immunogenic, and/or protective against a pathogenic species of ebolavirus. The dosage administered depends on the subject to be treated (e.g., the manner of administration and the age, body weight, capacity of the immune system, and general health of the subject being treated). The composition is administered in an amount to provide a sufficient level of expression that elicits an immune response without undue adverse physiological effects. Preferably, the composition of the invention is a heterologous viral vector that includes one or more polypeptides of the Lassa virus (e.g. Lassa virus prefusion glycoprotein and other forms of Lassa virus glycoprotein and large matrix protein; the vectors may also optionally express the Lassa virus nucleoprotein), or a nucleic acid molecule encoding one or more genes of Lassa virus, and is administered at a dosage of, e.g., between 1.0×10⁴ and 9.9×10¹² TCID₅₀ of the viral vector, preferably between 1.0×10⁵ TCID₅₀ and 1.0×10¹¹ TCID₅₀ pfu, more preferably between 1.0×10⁶ and 1.0×10¹⁰ TCID₅₀ pfu, or most preferably between 5.0×10⁶ and 5.0×10⁹ TCID₅₀. The composition may include, e.g., at least 5.0×10⁶ TCID₅₀ of the viral vector (e.g., 1.0×10⁸ TCID₅₀ of the viral vector). A physician or researcher can decide the appropriate amount and dosage regimen.

The composition of the method may include, e.g., between 1.0×10⁴ and 9.9×10¹² TCID₅₀ of the viral vector, preferably between 1.0×10⁵ TCID₅₀ and 1.0×10¹¹ TCID₅₀ pfu, more preferably between 1.0×10⁶ and 1.0×10¹⁰ TCID₅₀ pfu, or most preferably between 5.0×10⁶ and 5.0×10⁹ TCID₅₀. The composition may include, e.g., at least 5.0×10⁶ TCID₅₀ of the viral vector (e.g., 1.0×10⁸ TCID₅₀ of the viral vector). The method may include, e.g., administering the composition to the subject two or more times.

The invention also features a method of inducing an immune response to Lassa virus in a subject (e.g., a human) that includes administering to the subject an effective amount of a recombinant viral vector that encodes at least one gene from the Lassa virus (e.g., Lassa virus glycoprotein and large matrix protein; and optionally also express the Lassa virus nucleoprotein). The subject being treated may not have, but is at risk of developing, an infection by an arenavirus. Alternatively, the subject may already be infected with an arenavirus. The composition may be administered, e.g., by injection (e.g., intramuscular, intraarterial, intravascular, intravenous, intraperitoneal, or subcutaneous).

The term “effective amount” is meant the amount of a composition administered to improve, inhibit, or ameliorate a condition of a subject, or a symptom of a disorder, in a clinically relevant manner (e.g., improve, inhibit, or ameliorate infection by arenavirus or provide an effective immune response to infection by arenavirus). Any improvement in the subject is considered sufficient to achieve treatment. Preferably, an amount sufficient to treat is an amount that prevents the occurrence or one or more symptoms of ebolavirus, marburgvirus, or arenavirus infection or is an amount that reduces the severity of, or the length of time during which a subject suffers from, one or more symptoms of arenavirus infection (e.g., by at least 10%, 20%, or 30%, more preferably by at least 50%, 60%, or 70%, and most preferably by at least 80%, 90%, 95%, 99%, or more, relative to a control subject that is not treated with a composition of the invention). A sufficient amount of the pharmaceutical composition used to practice the methods described herein (e.g., the treatment of Lassa virus infection) varies depending upon the manner of administration and the age, body weight, and general health of the subject being treated. Ultimately, the prescribers or researchers will decide the appropriate amount and dosage.

It is important to note that the value of the present invention may never be demonstrated in terms of actual clinical benefit. Instead, it is likely that the value of the invention will be demonstrated in terms of success against a surrogate marker for protection. For an indication such as Lassa virus infection, in which it is impractical or unethical to attempt to measure clinical benefit of an intervention, the FDA's Accelerated Approval process allows approval of a new vaccine based on efficacy against a surrogate endpoint. Therefore, the value of the invention may lie in its ability to induce an immune response that constitutes a surrogate marker for protection.

Similarly, FDA may allow approval of vaccines against ebolaviruses, marburgviruses, or arenaviruses based on its Animal Rule. In this case, approval is achieved based on efficacy in animals. The value of the invention may lie in its ability to protect relevant animal species against infection with arenaviruses, thus providing adequate evidence to justify its approval.

The composition of the method may include, e.g., between 1.0×10⁴ and 9.9×10¹² TCID₅₀ of the viral vector, preferably between 1.0×10⁵ TCID₅₀ and 1.0×10¹¹ TCID₅₀ pfu, more preferably between 1.0×10⁶ and 1.0×10¹⁰ TCID₅₀ pfu, or most preferably between 5.0×10⁶ and 5.0×10⁹ TCID₅₀. The composition may include, e.g., at least 5.0×10⁶ TCID₅₀ of the viral vector (e.g., 1.0×10⁸ TCID₅₀ of the viral vector). The method may include, e.g., administering the composition two or more times.

In some instances, it may be desirable to combine the immunogenic arenavirus compositions of the present invention with immunogenic compositions which induce protective responses to other agents, particularly other viruses. For example, the vaccine compositions of the present invention can be administered simultaneously, separately or sequentially with other genetic immunization vaccines such as those for influenza (Ulmer, J. B. et al., Science 259:1745-1749 (1993); Raz, E. et al., PNAS (USA) 91:9519-9523 (1994)), malaria (Doolan, D. L. et al., J. Exp. Med. 183:1739-1746 (1996); Sedegah, M. et al., PNAS (USA) 91:9866-9870 (1994)), and tuberculosis (Tascon, R. C. et al., Nat. Med. 2:888-892 (1996)).

### Administration

As used herein, the term “administering” refers to a method of giving a dosage of a pharmaceutical composition of the invention to a subject. The compositions utilized in the methods described herein can be administered by a route selected from, e.g., parenteral, dermal, transdermal, ocular, inhalation, buccal, sublingual, perilingual, nasal, rectal, topical administration, and oral administration. Parenteral administration includes intravenous, intraperitoneal, subcutaneous, intraarterial, intravascular, and intramuscular administration. The preferred method of administration can vary depending on various factors (e.g., the components of the composition being administered, and the severity of the condition being treated).

Administration of the pharmaceutical compositions (e.g., vaccines) of the present invention can be by any of the routes known to one of skill in the art. Administration may be by, e.g., intramuscular injection. The compositions utilized in the methods described herein can also be administered by a route selected from, e.g., parenteral, dermal, transdermal, ocular, inhalation, buccal, sublingual, perilingual, nasal, rectal, topical administration, and oral administration. Parenteral administration includes intravenous, intraperitoneal, subcutaneous, and intramuscular administration. The preferred method of administration can vary depending on various factors, e.g., the components of the composition being administered, and the severity of the condition being treated.

In addition, single or multiple administrations of the compositions of the present invention may be given to a subject. For example, subjects who are particularly susceptible to ebolavirus infection may require multiple treatments to establish and/or maintain protection against the virus. Levels of induced immunity provided by the pharmaceutical compositions described herein can be monitored by, e.g., measuring amounts of neutralizing secretory and serum antibodies. The dosages may then be adjusted or repeated as necessary to maintain desired levels of protection against viral infection.

The claimed invention is further described by way of the following non-limiting examples. Further aspects and embodiments of the present invention will be apparent to those of ordinary skill in the art, in view of the above disclosure and following experimental exemplification, included by way of illustration and not limitation, and with reference to the attached figures.

### EXAMPLES

### Example 1. MVA Vaccine Vectors

This Example provides information on exemplary MVA vaccine vectors to lessen the burden of endemic LASV disease and to prevent future outbreaks. Vector GEO-LM01, a Modified Vaccinia Ankara (MVA)-vectored vaccine expressing LASV-like particles (VLPs) which provides a unique combination of advantages: (i) the immunological advantages of a live vector that elicits robust T cell and functional antibody (Ab) responses, (ii) the potent immunogenicity of VLPs, (iii) the inherent safety of the replication-deficient MVA vector, (iv) a simple and adjuvant-free presentation, and ideally single-dose protection. Indeed, as described below, GEO-LM01 produces VLPs that elicit strong and protective T cell responses after a single dose. There exists, however, a critical barrier in the field of LASV vaccine development: a vaccine is needed that has the properties of GEO-LM01 and that additionally can induce broadly neutralizing Ab (nAb) responses that protect against the multiple lineages of LASV. Further modifications of the GEO-LM01 vector are performed to create an immunogen that not only elicits a strong T cell response but also a lineage cross-reactive nAb response.

The sequence used for construction of GEO-LM01 GPC was based on the Josiah strain of LASV. To create MVA-VLP GEO-LM2.1, the GPC is stabilized in the pre-fusion conformation by modifying the Josiah sequence to introduce two cysteine residues (mutations R2070 and G3600) and the helix-breaking mutation E329P. Additional modifications are made using rational structural analysis techniques to ascertain the key sugar structures that contribute to the glycan shield that occludes easy access to the GPC-B tertiary epitope. Focusing on asparagine (N)-linked glycans with the goal of mutating the sequence of GPC at the identified N residues will provide additional vectors for use. In addition to providing a glycan shield, N-linked sugars also have stabilizing interactions with the peptide backbone.

Mutation of N99D and N119D, either alone or together increased detection of nAbs from LASV patient sera (Sommerstein, R. et al., PLoS Pathog 11(2015). The recombinant MVA-VLP vectors will encode GPC bearing these 2 mutations. Abs that bind GPC-B bury a substantial surface area on the GPC, particularly around the T-loop and HR2, which contains the four glycosylation sites present on LASV GPC (Hastie, K. M. et al., Science 356, 923-928 (2017). Two of these sites (N365 and N373) are critical for GPC-processing viral fitness Bonhomme, C. J. et al., PloS one 8, e53273 (2013); Eichler, R. et al., Virology journal 3, 41 (2006)). However, the remaining two (N390 and N395) are dispensable for both and are mutated to produce a modified GPC expression in MVA-VLP.

MVA-VLP-L2.1-4 are constructed utilizing the highly potent viral vector MVA with a high safety profile (Altenburg, A., Viruses 6, 27 (2014); Moss, B. et al., Advances in Experimental Medicine and Biology 397, 7-13 (1996)). The vaccines each produce two LASV proteins, GPC and Z proteins, which self-assemble into VLPs within the vaccinated host and serve as potent immunogens. The shuttle vectors used for construction of MVA-VLP-L2.1-4, produce stable vaccine inserts with high, but non-toxic, levels of expression. A recombinant MVA encoding LASV Z protein was produced by inserting sequences for Z into a restructured and modified deletion III between the A50R and B1R genes. The mutant GPC sequences described herein are placed between two essential genes of MVA (I8R and G1 L). All inserted sequences are codon optimized for MVA. Silent mutations are introduced to interrupt homo-polymer sequences (>4G/C and >4A/T) to reduce RNA polymerase errors that could lead to frameshifts. The sequences are edited for vaccinia-specific terminators to remove motifs that could lead to premature termination (Wyatt, L. S., et al., Vaccine 26, 486-493 (2008)). All vaccine inserts are placed under the modified H5 early/late vaccinia promoter as described previously (Wyatt, L. S., et al., Vaccine 14, 1451-1458 (1996)). The expression of full-length GPC is confirmed by Western blot. VLP formation is evaluated with thin section electron micrographs. The native conformation of GPC expressed on MVA-VLPs is assessed by immunostaining using LASV-specific GP1 and GP2 antibodies (a kind gift of Dr. James Robinson, Tulane University).

Table 2 lists MVA vaccine vectors.

TABLE 2
MVA vaccine vectors

| Vaccine | | Matrix |
|---|---|---|
| designation | GP sequence | protein sequence |
| GEO-LM01 | Wild type GP sequence | Optimized Z sequence for |
| | for LASV, Josiah strain | LASV, Josiah strain |
| GEO-LM2.1 | Optimized prefusion GP | Optimized Z sequence for |
| | sequence for LASV, | LASV, Josiah strain |
| | Josiah strain (mutations | |
| | R207C, G360C, E329P) | |
| GEO-LM2.2 | Optimized prefusion GP | Optimized Z sequence for |
| | sequence for LASV, | LASV, Josiah strain |
| | Josiah strain (mutations | |
| | R207C, G360C, E329P) | |
| | and N99D) | |
| GEO-LM2.3 | Optimized prefusion GP | Optimized Z sequence for |
| | sequence for LASV, | LASV, Josiah strain |
| | Josiah strain (mutations | |
| | R207C, G360C, E329P | |
| | and N119D) | |
| GEO-LM2.4 | Optimized prefusion GP | Optimized Z sequence for |
| | sequence for LASV, | LASV, Josiah strain |
| | Josiah strain (mutations | |
| | R207C, G360C, E329P, | |
| | N99D and N119D) | |
| GEO-LM2.5 | Optimized prefusion GP | Optimized Z sequence for |
| | sequence for LASV, | LASV, Josiah strain |
| | Josiah strain (mutations | |
| | R207C, G360C, E329P | |
| | and N390D) | |
| GEO-LM2.6 | Optimized prefusion GP | Optimized Z sequence for |
| | sequence for LASV, | LASV, Josiah strain |
| | Josiah strain (mutations | |
| | R207C, G360C, E329P | |
| | and N395D) | |
| GEO-LM2.7 | Optimized prefusion GP | Optimized Z sequence for |
| | sequence for LASV, | LASV, Josiah strain |
| | Josiah strain (mutations | |
| | R207C, G360C, E329P | |
| | and N390D and N395D) | |

In an exemplary embodiment, the vector expresses a modified Lassa virus glycoprotein complex having the following modifications.

TABLE 3
Modifications in Expressed Lassa Virus Glycoprotein
(Prefusion and/or Deglycosylation Mutant Glycoprotein)
Modification of Lassa Virus Glycoprotein expressed by MVA Vector

(a) point mutations R207C and G360C
(b) introduce a proline via an E329P mutation in the metastable region of
HR1 of RP2
(c) replace the native S1P GP1-GP2 cleavage site with a furin site (i.e.
RRLL to RRRR)
Optional additional mutations for deglycosylation mutants (alone or
combinations thereof)
(d) N99D
(e) N119D
(f) N390D
(g) N395D

### Example 2: MVA Vaccine Incorporating Further Mutations in Lassa Virus Sequences

In an exemplary embodiment, sequences from Lassa Virus (LARV) are prepared and optimized in shuttle plasmids and then the viral sequences are incorporated into an MVA vector. Such MVA vectors may be used individually as part of an administration protocol to elicit an immune response to Lassa Virus or as part of a multivalent vaccine composition having one or more MVA vectors expressing Lassa Virus antigens to elicit an immune response. Original Lassa GP and Z Sequences are obtained from Genbank (GenBank: JN650517.1 and JN650518.1) and optimized as described herein for insertion into MVA vectors.

Vaccine candidates are based upon the backbone of GEO-LM01, which has shown excellent T cell responses. MVA-VLP-L2.1 will introduce the R207C, G3600, and E329P mutations into GPC to create VLPs that express stabilized GPC that is locked in its prefusion state. In the other three candidates (MVA-VLP-L2.2, MVA-VLP-L2.3, and MVA-VLP-L2.4) we will introduce conservative point mutations into GPC to eliminate selected N-linked glycosylation motifs around the periphery of GPC-B, thereby further exposing this region to facilitate access of neutralizing Abs.

Additional mutations are optionally included in the glycoprotein sequence as shown in Table 4 below:

TABLE 4
Lassa Glycoprotein mutation table

| Changes | Mutation position |
|---|---|
| (Silent mutation) | on GP |
| T to C | 21 |
| T to C | 24 |
| T to C | 114 |
| A to G | 264 |
| T to C | 351 |
| A to G | 375 |
| A to G | 378 |
| A to G | 483 |
| T to C | 573 |
| A to G | 669 |
| T to C | 699 |
| T to C | 786 |
| A to G | 816 |
| A to G | 912 |
| A to G | 1056 |
| T to C | 1197 |
| A to G | 1251 |
| A to G | 1275 |
| T to C | 1308 |
| T to C | 1320 |
| A to G | 1353 |

The GEO-LM01 vector is a recombinant MVA designed to co-express a surface glycoprotein (GPC) and a matrix protein (Z) leading to the formation VLPs in the cells of the inoculated host. The GPC and Z sequences used in this vector were derived from the Josiah strain of LASV, which is a lineage IV strain. Expression of both Z protein and GPC that is cleaved into GP1 and GP2 subunits were demonstrated in cell lysates and supernatants of infected cells by Western blot (FIG. 4). Immunocytochemistry on infected cell monolayers demonstrated retention of both inserts in the viruses (FIG. 5). The assembly of VLPs from proteins expressed by GEO-LM01 was verified by electron microscopy (FIG. 6). Note the GPC spikes on the surface of virions (arrows).

We additionally assayed for the presence of binding Ab (bAb) to GPC in the serum of the immunized mice. Despite the excellent protection from death afforded by GEO-LM01, we found no statistically significant level of bAb above background (FIG. 7).

### Example 3: Immunogenic and Protective Potential of the MVA/Prefusion GP-VLP Vaccine

Immunogenicity and efficacy testing of MVA-VLP vectors is performed in a lethal mouse model, which uses ML29 virus for challenge. ML29 is a reassortant virus encoding the GPC and NP proteins of LASV (Josiah strain) and the L and Z proteins of Mopeia virus. The virus is uniformly lethal when administered by intracerebral (IC) inoculation into immunocompetent CBA/J mice. When administered by intraperitoneal (IP) inoculation, however, ML29 elicits a strong immune response that protects CBA/J mice from death upon subsequent IC challenge. To determine the best route of immunization, 4-6 week-old CBA/J mice (n=6) were immunized with 10⁷ TCID₅₀ of GEO-LM01 by IP, intramuscular (IM), or subcutaneous (SC) inoculation. Two groups of mice (n=6) were injected IP with ML29 (1,000 PFU) or saline, and served as positive and negative controls, respectively. Fourteen days later all mice were challenged by IC inoculation with 1,000 PFU of ML29 and monitored for weight change, morbidity, and mortality. Mice immunized with ML29 (IP) or with GEO-LM01 (IM) were 100% protected from lethal challenge and showed steady weights throughout the study, whereas mice administered saline alone uniformly died 8 days after lethal challenge (FIG. 4A, B). Mice immunized with GEO-LM01 by SC and IP administration showed less robust immunity to lethal challenge as seen in the more appreciable weight loss in these groups as well as the death of one animal in each group. A second study to measure immunogenicity and efficacy was initiated in the same model system, in this instance examining only two conditions: immunization with GEO-LM01 by IM inoculation and mock immunization with saline (n=10). Spleens were harvested from immunized animals (n=3) 11 days after a single administration of the vaccine. Antigen-specific T cell responses were measured by intracellular cytokine staining for IFNγ using LASV GPC peptides for stimulation. IFNγ expression was evident in both CD4+ and CD8+ T cells of immunized but not mock immunized mice. Some CD4+ T cells were also shown to be double positive for IFNγ and IL2, suggesting an expanding population. The remaining 7 animals from both groups were challenged on day 14 as in the first experiment. All vaccinated animals survived the challenge whereas all controls died by day 8 (data not shown) confirming the 100% single dose efficacy of GEO-LM01 observed in the previous study. The same is repeated for the other LASV-MVA vectors described herein.

The ML29 lethal challenge model in CBA/J mice is used to assess immunogenicity and efficacy of the newly constructed vaccines in a mouse model. All animals will be pre-bled for serum isolation. Five groups of mice will be vaccinated with 10⁷ TCID₅₀ (previously shown to fully protect animals, see FIG. 4) of the five vaccines or saline by IM administration. Three mice from each group are sacrificed 10 days after vaccination for splenectomy to measure T cell responses, as described below. On day 14 after vaccination, the remaining seven animals in each group are bled for serum prior to IC challenge by inoculation of 1,000 PFU ML29 virus. Following challenge, the animals are observed daily for signs of morbidity and mortality and will have weight and body temperature measured daily. Five days after challenge (day 17) three animals from each group are sacrificed for harvesting brains. A portion of the brain sample is quantified for ML29 virus by plaque assays using established methods to assess control of the infection in the brain. From the remainder of the brain samples, leukocytes are isolated and phenotyped by flow cytometry using CD3, CD4, and CD8 markers for T cells, CD19 for B cells, CD11b and GR1 for myeloid cells to assess the cellular immune response to infection in the brain.

Splenocytes are isolated from the spleens of animals sacrificed on day 10 to measure the T cell response in by intracellular cytokine staining (ICS) assay, reporting out the primary parameters of IL-2, IFNγ, and TNF production in CD4+ and CD8+ T cells as a result of LASV peptide stimulation. Pre-bleed and post-vaccination sera are analyzed by ELISA for the presence of bAb to recombinant GPC (MyBiosource) using the assay methods previously developed. The sera are assayed in for nAbs using a LASV pseudotype neutralization assay, utilizing a VSV/ΔG vector that expresses both LASV GPC and GFP to infect the U2OS target cell line. This method allows for rapid and robust assessment of neutralization by fluorescence microscopy.

### Example 4: Efficacy Testing of Selected Vaccine Candidate in Guinea Pigs Using LASV Lineages I-IV

In the field of LASV animal studies, female Hartley guinea pigs (HGP) are the standard small animal model for efficacy testing with live LASV as challenge virus. This model is used to test the down-selected MVA-VLP-L2 candidate side-by-side with GEO-LM01 following a prime-boost regimen and measuring humoral immunogenicity and efficacy across all four major LASV lineages. The prime-boost regimen is used in this study to amplify the immune response (especially Ab arm) such that differences between the vaccination conditions will be more readily apparent, which will be particularly important when challenging across numerous lineages. Twelve groups of six animals each will be immunized with GEO-LM01, the down-selected MVA-VLP-L2, or saline. Immunization follows the standard GeoVax protocol for guinea pig studies; in short, animals are inoculated by IM administration with 10⁸ TCID₅₀ of MVA-VLP-LASV (shown to fully protect with an MVA-VLP-EBOV, of each vaccine on days 0 and 28. The animals are bled on day 0 and every 14 days thereafter for collection of serum. On day 56 of the study, each individual set of immunized and control groups are inoculated with 1000 PFU of the four lineages of LASV. The particular strains used for each lineage are: the Pinneo strain (lineage I), strain 803213 (lineage II), the GA391 strain (lineage III), and the Josiah strain (lineage IV). Animals are observed for morbidity and mortality over the ensuing 28 days. On days 56-72, body temperatures are measured, the animals are weighed and scored for signs of pathology. Additionally, to measure viremia, serum samples are taken every 2 days over this period of time. On day 84 all surviving animals are sacrificed. Serum collected before challenge are assessed for the presence of bAb by ELISA.

### Example 5: Immunogenicity and Efficacy Testing in NHP

Before entering cGMP manufacturing for clinical testing of our vaccine, it is necessary to test efficacy in a large animal model. Cynomolgus macaques (Macaca fascicularis) are susceptible to LASV infection. This model is used to test both GEO-LM01 and MVA-VLP-L2 vectors. This study incorporates both (i) efficacy studies based on protection from morbidity and mortality following lethal challenge and (ii) an in-depth investigation of the cellular and humoral immunogenicity of both vaccines. The results of these studies will provide the data necessary for down-selecting to a single LASV vaccine to push toward human clinical trials.

The Josiah strain of LASV is uniformly lethal in cynomolgus macaques at a dose of 10⁴ PFU and is used to test the vaccines for efficacy. Three groups of five macaques that are 4 to 6 years old and weighing between 3 kg and 8 kg are immunized with GEO-LM01, MVA-VLP-L2, or mock-immunized with saline. A near-equal proportion of males and females are used in each group (2 males/3 females). In an ABSL-2 animal facility, the macaques are immunized by prime-boost regimen with vaccination on days 0 and 28 with a 10⁸ TCID₅₀ dose of each vaccine administered by IM route. On day 49 of the study the animals are transferred to an ABSL-4 containment facility. After seven days of acclimation to that facility (day 56) the animals are challenged by IM injection with 10⁴ PFU of LASV, Josiah strain. From previous published work it is known that in the absence of immunity, macaques succumb to LASV infection after challenge, as evidenced by fever (temperature over 104° F.) after about 3 days, followed by macular rashes, anorexia, and severe facial edema, and finally death within 10-15 days. For this study, the animals are observed twice daily after challenge for clinical signs. Temperature and weight measurements are taken daily. Serum samples are obtained on days 56, 58, 60, 62, 64, 66, and 84 for measurement of viremia by plaque assay.

Protection from death is the primary parameter for assessing efficacy in this model. Differences in the efficacy of the two vaccines may be further assess by measuring secondary parameters. In addition to death, macaques respond to LASV infection with noticeable changes in weight, body temperature, and overt clinical signs. Viremia after challenge is an important secondary parameter to measure.

The foregoing discussion discloses and describes merely exemplary embodiments of the present invention. One skilled in the art will readily recognize from such discussion, and from the accompanying drawings and claims, that various changes, modifications and variations can be made therein without departing from the spirit and scope of the invention as defined in the following claims.

All references cited herein are incorporated by reference in their entirety.

1. A recombinant modified vaccinia ankara (MVA) vector comprising a) a Lassa virus glycoprotein sequence selected from either a stabilized prefusion glycoprotein sequence or deglycosylation mutant glycoprotein sequence and b) an arenavirus matrix sequence, wherein both the glycoprotein sequence and the matrix sequence are under the control of one or more promoters compatible with poxvirus expression systems.

2. The recombinant vector of claim 1, wherein the glycoprotein sequence and the matrix sequence are inserted into one or more deletion sites of the MVA vector selected from I, II, III, IV, V or VI.

3. The recombinant vector of claim 1, wherein the glycoprotein sequence and the matrix sequence are inserted into the MVA vector in a natural deletion site, a modified natural deletion site or a site between essential or non-essential MVA genes.

4. The recombinant vector of claim 3, wherein the glycoprotein sequence and the matrix sequence are inserted into the same site.

5. The recombinant vector of claim 1, wherein the glycoprotein sequence and the matrix sequence are inserted into different sites.

6. The recombinant vector of claim 1, wherein the glycoprotein sequence is inserted into a site between two essential and highly conserved MVA genes, and the matrix sequence is inserted into a restructured and modified deletion site III.

7. The recombinant vector of claim 1, wherein the glycoprotein sequence is inserted between MVA genes I8R and G1L

8. The recombinant vector of claim 1, wherein the promoter is selected from the group consisting of Pm2H5, Psyn II, mH5 promoters or combinations thereof.

10. The recombinant vector of claim 1, wherein the prefusion glycoprotein and the matrix protein are expressed and assemble into VLPs.

11. The recombinant vector of claim 1, wherein the matrix protein is VP40.

12. The recombinant vector of claim 1, wherein the glycoprotein sequence and the matrix sequence are from the same species.

13. The recombinant vector of claim 1, wherein the glycoprotein sequence and the matrix sequence are from different species.

14. The recombinant vector of claim 1, wherein the matrix sequence is LASV Z.

15. The recombinant vector of claim 14, further comprising the NP sequence of LASV.

16. A pharmaceutical composition comprising at least one recombinant MVA vector and a pharmaceutically acceptable carrier, wherein the recombinant MVA vector comprises (i) a Lassa virus glycoprotein sequence selected from either a stabilized prefusion glycoprotein sequence or deglycosylation mutant glycoprotein sequence and (ii) a matrix sequence from virus selected from the group consisting of an ebolavirus, a Marburgvirus, or arenavirus, wherein both the prefusion glycoprotein sequence and matrix sequence are under the control of promoters compatible with poxvirus expression systems.

17. The pharmaceutical composition of claim 16, formulated for intraperitoneal, intramuscular, intradermal, epidermal, mucosal or intravenous administration.

18. The pharmaceutical composition of claim 16, comprising two recombinant MVA vectors, wherein the first recombinant MVA vector comprises (i) a Lassa virus prefusion glycoprotein sequence and (ii) a matrix sequence from a virus selected from the group consisting of an ebolavirus, a Marburgvirus or an arenavirus,
and wherein the second recombinant MVA vector comprises (i) a wild type Lassa virus glycoprotein sequence and (ii) a matrix sequence from a virus selected from the group consisting of an ebolavirus, a Marburgvirus or an arenavirus,
wherein the glycoprotein sequence and matrix sequences of the first recombinant MVA vector are different than the glycoprotein sequence and matrix sequence of the second recombinant MVA vector.

19. A method of inducing an immune response in a subject in need thereof, said method comprising administering at least one recombinant MVA vector to the subject in an amount sufficient to induce an immune response,
wherein the recombinant MVA vector comprises (i) a Lassa virus glycoprotein sequence selected from either a stabilized prefusion glycoprotein sequence or deglycosylation mutant glycoprotein sequence and (ii) a matrix sequence from a virus selected from the group consisting of an ebolavirus, a marburgvirus, or arenavirus, wherein both the glycoprotein sequence and the matrix sequence are operably linked to promoters compatible with poxvirus expression systems.

20. The method of claim 19, wherein the immune response is selected from a humoral immune response, a cellular immune response or a combination thereof.

21. The method of claim 19, wherein the immune response comprises production of binding antibodies or neutralizing antibodies against the arenavirus.

22. The method of claim 19, wherein the immune response comprises production of non-neutralizing antibodies against the arenavirus.

23. The method of claim 19, wherein the immune response comprises production of a cell-mediated immune response against the arenavirus.

24. A method of preventing an infection by an arenavirus in a subject in need thereof, said method comprising administering at least one recombinant MVA vector to the subject in in a prophylactically effective amount,
wherein the recombinant MVA vector comprises (i) a Lassa virus glycoprotein sequence selected from either a stabilized prefusion glycoprotein sequence or deglycosylation mutant glycoprotein sequence and (ii) a matrix sequence from a virus selected from the group consisting of an ebolavirus, a marburgvirus, or arenavirus, wherein both the prefusion glycoprotein sequence and the matrix sequence are operably linked to a promoter compatible with poxvirus expression systems.

25. The method of claim 24, wherein the arenavirus is Lassa virus, and the method prevents infection by a Lassa virus.

26. A method of inducing an immune response to an arenavirus in a subject in need thereof, said method comprising administering a recombinant MVA vector to the subject in an effective amount to induce an immune response,
wherein the recombinant MVA vector comprises (i) a Lassa virus glycoprotein sequence selected from either a stabilized prefusion glycoprotein sequence or deglycosylation mutant glycoprotein sequence and (ii) a matrix sequence from a virus selected from the group consisting of an ebolavirus, a marburgvirus, or arenavirus, wherein both the prefusion glycoprotein sequence and the matrix sequence are operably linked to a promoter compatible with poxvirus expression systems.

27. A method of treating infection by an arenavirus in a subject in need thereof, said method comprising administering a recombinant MVA vector in a therapeutically effective amount to said subject,
wherein the recombinant MVA vector comprises (i) a Lassa virus glycoprotein sequence and (ii) a matrix sequence from a virus selected from the group consisting of an ebolavirus, a marburgvirus, or arenavirus, wherein both the prefusion glycoprotein sequence and matrix sequence are operably linked to a promoter compatible with poxvirus expression systems.

28. The method of claim 27, wherein the subject was recently exposed to an arenavirus but not yet symptomatic.

29. The method of claim 27, wherein the subject was exposed to an arenavirus but exhibits minimal symptoms of infections.

30. The method of claim 27, wherein the method results in amelioration of at least one symptom of infection.

31. The method of claim 27, wherein the method results in reduction or elimination of the subject's ability to transmit the infection to an uninfected subject.

32. The method of claim 27, wherein the method prevents or ameliorates infections resulting from one or more species of arenavirus.


SEQUENCE LISTING (18 sequences)

SEQ ID NO: 1 - DNA, 1479 bp, Lassa virus
atgggacaaatagtaacattcttccaagaagtaccacatgtaattgaagaagtaatgaatattgtactaattgcgctatctgtactagcggtattgaaaggattgtataatttcgcgacatgtggactagtaggactagttacatttctactactatgtggaagatcttgtacaacttctttgtataaaggagtatatgaactacaaacactagaattgaatatggaaactctaaatatgacaatgcctctatcatgtacaaagaataattctcatcattatattatggttggaaatgaaacaggactagaactaacactaacaaatacttctattattaatcataaattctgtaatctatctgatgcgcataagaagaatctatatgatcatgcgctaatgtctattatttctacatttcatctatctattccaaactttaatcaatatgaagctatgtcttgtgactttaatggtggaaagatttctgtacaatataatctaagtcattcttatgcgggagatgcggcgaatcattgtggaacagtagcgaatggtgtactacaaactttcatgagaatggcgtggggaggatcttatattgcgctagattctggaagaggaaattgggattgtattatgacatcttatcaatatctaattattcagaatacaacatgggaagatcattgtcaattctctagaccatctccaataggatatctaggactactatctcaaagaacaagagatatatatattagtagaagattgctaggaactttcacatggacactatctgattctgaaggaaaggatacacctggaggatattgtctaacaagatggatgctaattgaagcggaattgaaatgttttggaaatactgcggtagcgaaatgtaatgaaaagcatgatgaagaattttgtgatatgctaagactatttgactttaataaacaagcgattcaaagattgaaagcggaagcgcaaatgagtattcaattgataaataaagcggttaatgctttgattaatgatcaactaattatgaagaatcatctaagagatattatgggaattccatattgtaattatagtaaatattggtatctaaatcatacaacaactggaagaacatctctaccaaaatgttggctagtatctaatggatcttatctaaatgaaacacatttctctgatgatattgaacaacaagcggataatatgattacagaaatgctacaaaaggaatatatggaaagacaaggaaagacaccactaggattggtagatctatttgttttctctacatctttctatctaattagtatatttctacatctagtaaagattccaacacatagacatatagtaggaaaatcttgtccaaaaccacatagattgaatcatatgggaatatgttcttgtggattgtataaacaaccaggtgtaccagttaaatggaaaagataataa

SEQ ID NO: 2 - protein, 491 aa, Lassa virus
MGQIVTFFQEVPHVIEEVMNIVLIALSVLAVLKGLYNFATCGLVGLVTFLLLCGRSCTTSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGRGNWDCIMTSYQYLIIQNTTWEDHCQFSRPSPIGYLGLLSQRTRDIYISRRLLGTFTWTLSDSEGKDTPGGYCLTRWMLIEAELKCFGNTAVAKCNEKHDEEFCDMLRLFDFNKQAIQRLKAEAQMSIQLINKAVNALINDQLIMKNHLRDIMGIPYCNYSKYWYLNHTTTGRTSLPKCWLVSNGSYLNETHFSDDIEQQADNMITEMLQKEYMERQGKTPLGLVDLFVFSTSFYLISIFLHLVKIPTHRHIVGKSCPKPHRLNHMGICSCGLYKQPGVPVKWKR

SEQ ID NO: 3 - DNA, 1476 bp, Lassa virus
atgggacaaatagtaacattcttccaagaagtaccacatgtaattgaagaagtaatgaatattgtactaattgcgctatctgtactagcggtattgaaaggattgtataatttcgcgacatgtggactagtaggactagttacatttctactactatgtggaagatcttgtacaacttctttgtataaaggagtatatgaactacaaacactagaattgaatatggaaactctaaatatgacaatgcctctatcatgtacaaagaataattctcatcattatattatggttggagacgaaacaggactagaactaacactaacaaatacttctattattaatcataaattctgtaatctatctgatgcgcataagaagaatctatatgatcatgcgctaatgtctattatttctacatttcatctatctattccaaactttaatcaatatgaagctatgtcttgtgactttaatggtggaaagatttctgtacaatataatctaagtcattcttatgcgggagatgcggcgaatcattgtggaacagtagcgaatggtgtactacaaactttcatgagaatggcgtggggaggatcttatattgcgctagattctggaagaggaaattgggattgtattatgacatcttatcaatatctaattattcagaatacaacatgggaagatcattgtcaattctctagaccatctccaataggatatctaggactactatctcaaagaacaagagatatatatattagtagaagattgctaggaactttcacatggacactatctgattctgaaggaaaggatacacctggaggatattgtctaacaagatggatgctaattgaagcggaattgaaatgttttggaaatactgcggtagcgaaatgtaatgaaaagcatgatgaagaattttgtgatatgctaagactatttgactttaataaacaagcgattcaaagattgaaagcggaagcgcaaatgagtattcaattgataaataaagcggttaatgctttgattaatgatcaactaattatgaagaatcatctaagagatattatgggaattccatattgtaattatagtaaatattggtatctaaatcatacaacaactggaagaacatctctaccaaaatgttggctagtatctaatggatcttatctaaatgaaacacatttctctgatgatattgaacaacaagcggataatatgattacagaaatgctacaaaaggaatatatggaaagacaaggaaagacaccactaggattggtagatctatttgttttctctacatctttctatctaattagtatatttctacatctagtaaagattccaacacatagacatatagtaggaaaatcttgtccaaaaccacatagattgaatcatatgggaatatgttcttgtggattgtataaacaaccaggtgtaccagttaaatggaaaagataa

SEQ ID NO: 4 - protein, 491 aa, Lassa virus
MGQIVTFFQEVPHVIEEVMNIVLIALSVLAVLKGLYNFATCGLVGLVTFLLLCGRSCTTSLYKGVYELQTLELNMETLNMTMPLSCTKNNSHHYIMVGDETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGRGNWDCIMTSYQYLIIQNTTWEDHCQFSRPSPIGYLGLLSQRTRDIYISRRLLGTFTWTLSDSEGKDTPGGYCLTRWMLIEAELKCFGNTAVAKCNEKHDEEFCDMLRLFDFNKQAIQRLKAEAQMSIQLINKAVNALINDQLIMKNHLRDIMGIPYCNYSKYWYLNHTTTGRTSLPKCWLVSNGSYLNETHFSDDIEQQADNMITEMLQKEYMERQGKTPLGLVDLFVFSTSFYLISIFLHLVKIPTHRHIVGKSCPKPHRLNHMGICSCGLYKQPGVPVKWKR

SEQ ID NO: 5 - DNA, 1476 bp, Lassa virus
atgggacaaatagtaacattcttccaagaagtaccacatgtaattgaagaagtaatgaatattgtactaattgcgctatctgtactagcggtattgaaaggattgtataatttcgcgacatgtggactagtaggactagttacatttctactactatgtggaagatcttgtacaacttctttgtataaaggagtatatgaactacaaacactagaattgaatatggaaactctaaatatgacaatgcctctatcatgtacaaagaataattctcatcattatattatggttggaaatgaaacaggactagaactaacactaacaaatacttctattattaatcataaattctgtgacctatctgatgcgcataagaagaatctatatgatcatgcgctaatgtctattatttctacatttcatctatctattccaaactttaatcaatatgaagctatgtcttgtgactttaatggtggaaagatttctgtacaatataatctaagtcattcttatgcgggagatgcggcgaatcattgtggaacagtagcgaatggtgtactacaaactttcatgagaatggcgtggggaggatcttatattgcgctagattctggaagaggaaattgggattgtattatgacatcttatcaatatctaattattcagaatacaacatgggaagatcattgtcaattctctagaccatctccaataggatatctaggactactatctcaaagaacaagagatatatatattagtagaagattgctaggaactttcacatggacactatctgattctgaaggaaaggatacacctggaggatattgtctaacaagatggatgctaattgaagcggaattgaaatgttttggaaatactgcggtagcgaaatgtaatgaaaagcatgatgaagaattttgtgatatgctaagactatttgactttaataaacaagcgattcaaagattgaaagcggaagcgcaaatgagtattcaattgataaataaagcggttaatgctttgattaatgatcaactaattatgaagaatcatctaagagatattatgggaattccatattgtaattatagtaaatattggtatctaaatcatacaacaactggaagaacatctctaccaaaatgttggctagtatctaatggatcttatctaaatgaaacacatttctctgatgatattgaacaacaagcggataatatgattacagaaatgctacaaaaggaatatatggaaagacaaggaaagacaccactaggattggtagatctatttgttttctctacatctttctatctaattagtatatttctacatctagtaaagattccaacacatagacatatagtaggaaaatcttgtccaaaaccacatagattgaatcatatgggaatatgttcttgtggattgtataaacaaccaggtgtaccagttaaatggaaaagataa

SEQ ID NO: 6 - protein, 491 aa, Lassa virus
MGQIVTFFQEVPHVIEEVMNIVLIALSVLAVLKGLYNFATCGLVGLVTFLLLCGRSCTTSLYKGVYELQTLELNMETLNMTMPLSCTKNNSHHYIMVGNETGLELTLTNTSIINHKFCDLSDAHKKNLYDHALMSIISTFHLSIPNFNQYEAMSCDFNGGKISVQYNLSHSYAGDAANHCGTVANGVLQTFMRMAWGGSYIALDSGRGNWDCIMTSYQYLIIQNTTWEDHCQFSRPSPIGYLGLLSQRTRDIYISRRLLGTFTWTLSDSEGKDTPGGYCLTRWMLIEAELKCFGNTAVAKCNEKHDEEFCDMLRLFDFNKQAIQRLKAEAQMSIQLINKAVNALINDQLIMKNHLRDIMGIPYCNYSKYWYLNHTTTGRTSLPKCWLVSNGSYLNETHFSDDIEQQADNMITEMLQKEYMERQGKTPLGLVDLFVFSTSFYLISIFLHLVKIPTHRHIVGKSCPKPHRLNHMGICSCGLYKQPGVPVKWKR

SEQ ID NO: 7 - DNA, 1479 bp, Lassa virus
atgggacaaatagtaacattcttccaagaagtaccacatgtaattgaagaagtaatgaatattgtactaattgcgctatctgtactagcggtattgaaaggattgtataatttcgcgacatgtggactagtaggactagttacatttctactactatgtggaagatcttgtacaacttctttgtataaaggagtatatgaactacaaacactagaattgaatatggaaactctaaatatgacaatgcctctatcatgtacaaagaataattctcatcattatattatggttggagacgaaacaggactagaactaacactaacaaatacttctattattaatcataaattctgtgacctatctgatgcgcataagaagaatctatatgatcatgcgctaatgtctattatttctacatttcatctatctattccaaactttaatcaatatgaagctatgtcttgtgactttaatggtggaaagatttctgtacaatataatctaagtcattcttatgcgggagatgcggcgaatcattgtggaacagtagcgaatggtgtactacaaactttcatgagaatggcgtggggaggatcttatattgcgctagattctggaagaggaaattgggattgtattatgacatcttatcaatatctaattattcagaatacaacatgggaagatcattgtcaattctctagaccatctccaataggatatctaggactactatctcaaagaacaagagatatatatattagtagaagattgctaggaactttcacatggacactatctgattctgaaggaaaggatacacctggaggatattgtctaacaagatggatgctaattgaagcggaattgaaatgttttggaaatactgcggtagcgaaatgtaatgaaaagcatgatgaagaattttgtgatatgctaagactatttgactttaataaacaagcgattcaaagattgaaagcggaagcgcaaatgagtattcaattgataaataaagcggttaatgctttgattaatgatcaactaattatgaagaatcatctaagagatattatgggaattccatattgtaattatagtaaatattggtatctaaatcatacaacaactggaagaacatctctaccaaaatgttggctagtatctaatggatcttatctaaatgaaacacatttctctgatgatattgaacaacaagcggataatatgattacagaaatgctacaaaaggaatatatggaaagacaaggaaagacaccactaggattggtagatctatttgttttctctacatctttctatctaattagtatatttctacatctagtaaagattccaacacatagacatatagtaggaaaatcttgtccaaaaccacatagattgaatcatatgggaatatgttcttgtggattgtataaacaaccaggtgtaccagttaaatggaaaagataataa

SEQ ID NO: 8 - protein, 491 aa, Lassa virus
MGQIVTFFQEVPHVIEEVMNIVLIALSVLAVLKGLYNFATCGLVGLVTFLLLCGRSCTTSLYKGVYELQTLELNMETLNMTMPLSCTKNNSHHYIMVGDETGLELTLTNTSIINHKFCDLSDAHKKNLYDHALMSIISTFHLSIPNFNQYEAMSCDFNGGKISVQYNLSHSYAGDAANHCGTVANGVLQTFMRMAWGGSYIALDSGRGNWDCIMTSYQYLIIQNTTWEDHCQFSRPSPIGYLGLLSQRTRDIYISRRLLGTFTWTLSDSEGKDTPGGYCLTRWMLIEAELKCFGNTAVAKCNEKHDEEFCDMLRLFDFNKQAIQRLKAEAQMSIQLINKAVNALINDQLIMKNHLRDIMGIPYCNYSKYWYLNHTTTGRTSLPKCWLVSNGSYLNETHFSDDIEQQADNMITEMLQKEYMERQGKTPLGLVDLFVFSTSFYLISIFLHLVKIPTHRHIVGKSCPKPHRLNHMGICSCGLYKQPGVPVKWKR

SEQ ID NO: 9 - DNA, 1476 bp, Lassa virus
atgggacaaatagtaacattcttccaagaagtaccacatgtaattgaagaagtaatgaatattgtactaattgcgctatctgtactagcggtattgaaaggattgtataatttcgcgacatgtggactagtaggactagttacatttctactactatgtggaagatcttgtacaacttctttgtataaaggagtatatgaactacaaacactagaattgaatatggaaactctaaatatgacaatgcctctatcatgtacaaagaataattctcatcattatattatggttggaaatgaaacaggactagaactaacactaacaaatacttctattattaatcataaattctgtaatctatctgatgcgcataagaagaatctatatgatcatgcgctaatgtctattatttctacatttcatctatctattccaaactttaatcaatatgaagctatgtcttgtgactttaatggtggaaagatttctgtacaatataatctaagtcattcttatgcgggagatgcggcgaatcattgtggaacagtagcgaatggtgtactacaaactttcatgagaatggcgtggggaggatcttatattgcgctagattctggctgtggaaattgggattgtattatgacatcttatcaatatctaattattcagaatacaacatgggaagatcattgtcaattctctagaccatctccaataggatatctaggactactatctcaaagaacaagagatatatatattagtagaagattgctaggaactttcacatggacactatctgattctgaaggaaaggatacacctggaggatattgtctaacaagatggatgctaattgaagcggaattgaaatgttttggaaatactgcggtagcgaaatgtaatgaaaagcatgatgaagaattttgtgatatgctaagactatttgactttaataaacaagcgattcaaagattgaaagcgcctgcgcaaatgagtattcaattgataaataaagcggttaatgctttgattaatgatcaactaattatgaagaatcatctaagagatattatgtgcattccatattgtaattatagtaaatattggtatctaaatcatacaacaactggaagaacatctctaccaaaatgttggctagtatctaatggatcttatctaaatgaaacacatttctctgatgatattgaacaacaagcggataatatgattacagaaatgctacaaaaggaatatatggaaagacaaggaaagacaccactaggattggtagatctatttgttttctctacatctttctatctaattagtatatttctacatctagtaaagattccaacacatagacatatagtaggaaaatcttgtccaaaaccacatagattgaatcatatgggaatatgttcttgtggattgtataaacaaccaggtgtaccagttaaatggaaaagataa

SEQ ID NO: 10 - protein, 491 aa, Lassa virus
MGQIVTFFQEVPHVIEEVMNIVLIALSVLAVLKGLYNFATCGLVGLVTFLLLCGRSCTTSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGCGNWDCIMTSYQYLIIQNTTWEDHCQFSRPSPIGYLGLLSQRTRDIYISRRLLGTFTWTLSDSEGKDTPGGYCLTRWMLIEAELKCFGNTAVAKCNEKHDEEFCDMLRLFDFNKQAIQRLKAPAQMSIQLINKAVNALINDQLIMKNHLRDIMCIPYCNYSKYWYLNHTTTGRTSLPKCWLVSNGSYLNETHFSDDIEQQADNMITEMLQKEYMERQGKTPLGLVDLFVFSTSFYLISIFLHLVKIPTHRHIVGKSCPKPHRLNHMGICSCGLYKQPGVPVKWKR

SEQ ID NO: 11 - DNA, 1476 bp, Lassa virus
atgggacaaatagtaacattcttccaagaagtaccacatgtaattgaagaagtaatgaatattgtactaattgcgctatctgtactagcggtattgaaaggattgtataatttcgcgacatgtggactagtaggactagttacatttctactactatgtggaagatcttgtacaacttctttgtataaaggagtatatgaactacaaacactagaattgaatatggaaactctaaatatgacaatgcctctatcatgtacaaagaataattctcatcattatattatggttggagacgaaacaggactagaactaacactaacaaatacttctattattaatcataaattctgtaatctatctgatgcgcataagaagaatctatatgatcatgcgctaatgtctattatttctacatttcatctatctattccaaactttaatcaatatgaagctatgtcttgtgactttaatggtggaaagatttctgtacaatataatctaagtcattcttatgcgggagatgcggcgaatcattgtggaacagtagcgaatggtgtactacaaactttcatgagaatggcgtggggaggatcttatattgcgctagattctggctgtggaaattgggattgtattatgacatcttatcaatatctaattattcagaatacaacatgggaagatcattgtcaattctctagaccatctccaataggatatctaggactactatctcaaagaacaagagatatatatattagtagaagattgctaggaactttcacatggacactatctgattctgaaggaaaggatacacctggaggatattgtctaacaagatggatgctaattgaagcggaattgaaatgttttggaaatactgcggtagcgaaatgtaatgaaaagcatgatgaagaattttgtgatatgctaagactatttgactttaataaacaagcgattcaaagattgaaagcgcctgcgcaaatgagtattcaattgataaataaagcggttaatgctttgattaatgatcaactaattatgaagaatcatctaagagatattatgtgcattccatattgtaattatagtaaatattggtatctaaatcatacaacaactggaagaacatctctaccaaaatgttggctagtatctaatggatcttatctaaatgaaacacatttctctgatgatattgaacaacaagcggataatatgattacagaaatgctacaaaaggaatatatggaaagacaaggaaagacaccactaggattggtagatctatttgttttctctacatctttctatctaattagtatatttctacatctagtaaagattccaacacatagacatatagtaggaaaatcttgtccaaaaccacatagattgaatcatatgggaatatgttcttgtggattgtataaacaaccaggtgtaccagttaaatggaaaagataa

SEQ ID NO: 12 - protein, 491 aa, Lassa virus
MGQIVTFFQEVPHVIEEVMNIVLIALSVLAVLKGLYNFATCGLVGLVTFLLLCGRSCTTSLYKGVYELQTLELNMETLNMTMPLSCTKNNSHHYIMVGDETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGCGNWDCIMTSYQYLIIQNTTWEDHCQFSRPSPIGYLGLLSQRTRDIYISRRLLGTFTWTLSDSEGKDTPGGYCLTRWMLIEAELKCFGNTAVAKCNEKHDEEFCDMLRLFDFNKQAIQRLKAPAQMSIQLINKAVNALINDQLIMKNHLRDIMCIPYCNYSKYWYLNHTTTGRTSLPKCWLVSNGSYLNETHFSDDIEQQADNMITEMLQKEYMERQGKTPLGLVDLFVFSTSFYLISIFLHLVKIPTHRHIVGKSCPKPHRLNHMGICSCGLYKQPGVPVKWKR

SEQ ID NO: 13 - DNA, 1476 bp, Lassa virus
atgggacaaatagtaacattcttccaagaagtaccacatgtaattgaagaagtaatgaatattgtactaattgcgctatctgtactagcggtattgaaaggattgtataatttcgcgacatgtggactagtaggactagttacatttctactactatgtggaagatcttgtacaacttctttgtataaaggagtatatgaactacaaacactagaattgaatatggaaactctaaatatgacaatgcctctatcatgtacaaagaataattctcatcattatattatggttggaaatgaaacaggactagaactaacactaacaaatacttctattattaatcataaattctgtgacctatctgatgcgcataagaagaatctatatgatcatgcgctaatgtctattatttctacatttcatctatctattccaaactttaatcaatatgaagctatgtcttgtgactttaatggtggaaagatttctgtacaatataatctaagtcattcttatgcgggagatgcggcgaatcattgtggaacagtagcgaatggtgtactacaaactttcatgagaatggcgtggggaggatcttatattgcgctagattctggctgtggaaattgggattgtattatgacatcttatcaatatctaattattcagaatacaacatgggaagatcattgtcaattctctagaccatctccaataggatatctaggactactatctcaaagaacaagagatatatatattagtagaagattgctaggaactttcacatggacactatctgattctgaaggaaaggatacacctggaggatattgtctaacaagatggatgctaattgaagcggaattgaaatgttttggaaatactgcggtagcgaaatgtaatgaaaagcatgatgaagaattttgtgatatgctaagactatttgactttaataaacaagcgattcaaagattgaaagcgcctgcgcaaatgagtattcaattgataaataaagcggttaatgctttgattaatgatcaactaattatgaagaatcatctaagagatattatgtgcattccatattgtaattatagtaaatattggtatctaaatcatacaacaactggaagaacatctctaccaaaatgttggctagtatctaatggatcttatctaaatgaaacacatttctctgatgatattgaacaacaagcggataatatgattacagaaatgctacaaaaggaatatatggaaagacaaggaaagacaccactaggattggtagatctatttgttttctctacatctttctatctaattagtatatttctacatctagtaaagattccaacacatagacatatagtaggaaaatcttgtccaaaaccacatagattgaatcatatgggaatatgttcttgtggattgtataaacaaccaggtgtaccagttaaatggaaaagataa

SEQ ID NO: 14 - protein, 491 aa, Lassa virus
MGQIVTFFQEVPHVIEEVMNIVLIALSVLAVLKGLYNFATCGLVGLVTFLLLCGRSCTTSLYKGVYELQTLELNMETLNMTMPLSCTKNNSHHYIMVGNETGLELTLTNTSIINHKFCDLSDAHKKNLYDHALMSIISTFHLSIPNFNQYEAMSCDFNGGKISVQYNLSHSYAGDAANHCGTVANGVLQTFMRMAWGGSYIALDSGCGNWDCIMTSYQYLIIQNTTWEDHCQFSRPSPIGYLGLLSQRTRDIYISRRLLGTFTWTLSDSEGKDTPGGYCLTRWMLIEAELKCFGNTAVAKCNEKHDEEFCDMLRLFDFNKQAIQRLKAPAQMSIQLINKAVNALINDQLIMKNHLRDIMCIPYCNYSKYWYLNHTTTGRTSLPKCWLVSNGSYLNETHFSDDIEQQADNMITEMLQKEYMERQGKTPLGLVDLFVFSTSFYLISIFLHLVKIPTHRHIVGKSCPKPHRLNHMGICSCGLYKQPGVPVKWKR

SEQ ID NO: 15 - DNA, 1476 bp, Lassa virus
atgggacaaatagtaacattcttccaagaagtaccacatgtaattgaagaagtaatgaatattgtactaattgcgctatctgtactagcggtattgaaaggattgtataatttcgcgacatgtggactagtaggactagttacatttctactactatgtggaagatcttgtacaacttctttgtataaaggagtatatgaactacaaacactagaattgaatatggaaactctaaatatgacaatgcctctatcatgtacaaagaataattctcatcattatattatggttggagacgaaacaggactagaactaacactaacaaatacttctattattaatcataaattctgtgacctatctgatgcgcataagaagaatctatatgatcatgcgctaatgtctattatttctacatttcatctatctattccaaactttaatcaatatgaagctatgtcttgtgactttaatggtggaaagatttctgtacaatataatctaagtcattcttatgcgggagatgcggcgaatcattgtggaacagtagcgaatggtgtactacaaactttcatgagaatggcgtggggaggatcttatattgcgctagattctggctgtggaaattgggattgtattatgacatcttatcaatatctaattattcagaatacaacatgggaagatcattgtcaattctctagaccatctccaataggatatctaggactactatctcaaagaacaagagatatatatattagtagaagattgctaggaactttcacatggacactatctgattctgaaggaaaggatacacctggaggatattgtctaacaagatggatgctaattgaagcggaattgaaatgttttggaaatactgcggtagcgaaatgtaatgaaaagcatgatgaagaattttgtgatatgctaagactatttgactttaataaacaagcgattcaaagattgaaagcgcctgcgcaaatgagtattcaattgataaataaagcggttaatgctttgattaatgatcaactaattatgaagaatcatctaagagatattatgtgcattccatattgtaattatagtaaatattggtatctaaatcatacaacaactggaagaacatctctaccaaaatgttggctagtatctaatggatcttatctaaatgaaacacatttctctgatgatattgaacaacaagcggataatatgattacagaaatgctacaaaaggaatatatggaaagacaaggaaagacaccactaggattggtagatctatttgttttctctacatctttctatctaattagtatatttctacatctagtaaagattccaacacatagacatatagtaggaaaatcttgtccaaaaccacatagattgaatcatatgggaatatgttcttgtggattgtataaacaaccaggtgtaccagttaaatggaaaagataa

SEQ ID NO: 16 - protein, 491 aa, Lassa virus
MGQIVTFFQEVPHVIEEVMNIVLIALSVLAVLKGLYNFATCGLVGLVTFLLLCGRSCTTSLYKGVYELQTLELNMETLNMTMPLSCTKNNSHHYIMVGDETGLELTLTNTSIINHKFCDLSDAHKKNLYDHALMSIISTFHLSIPNFNQYEAMSCDFNGGKISVQYNLSHSYAGDAANHCGTVANGVLQTFMRMAWGGSYIALDSGCGNWDCIMTSYQYLIIQNTTWEDHCQFSRPSPIGYLGLLSQRTRDIYISRRLLGTFTWTLSDSEGKDTPGGYCLTRWMLIEAELKCFGNTAVAKCNEKHDEEFCDMLRLFDFNKQAIQRLKAPAQMSIQLINKAVNALINDQLIMKNHLRDIMCIPYCNYSKYWYLNHTTTGRTSLPKCWLVSNGSYLNETHFSDDIEQQADNMITEMLQKEYMERQGKTPLGLVDLFVFSTSFYLISIFLHLVKIPTHRHIVGKSCPKPHRLNHMGICSCGLYKQPGVPVKWKR

SEQ ID NO: 17 - DNA, 303 bp, Lassa virus
atgggaaataaacaagcgaaagcgccagaatctaaagattctccaagagcgagtctaattccagatgcgacacatctaggaccacaattttgtaaatcttgttggtttgaaaataaaggactagtagaatgtaataatcattatctatgtctaaattgtctaacactactactatctgtatctaatagatgtccaatatgcaaaatgccactaccaacaaaactaagaccatctgctgctccaacagcgccaccaacaggtgctgctgattctattagaccaccaccatattctccataataa

SEQ ID NO: 18 - protein, 99 aa, Lassa virus
MGNKQAKAPESKDSPRASLIPDATHLGPQFCKSCWFENKGLVECNNHYLCLNCLTLLLSVSNRCPICKMPLPTKLRPSAAPTAPPTGAADSIRPPPYSP